SEQUENCE LISTING (2 sequences)

SEQ ID NO: 1 - protein, 1055 aa, Aggregatibacter actinomycetemcomitans
MATTSLLNTKQQAAQFANSVADRAKENIDAAKEQLQKALDKLGKTGKKLTLYIKNYKKGNGLTALIKAAQKLGIEVYHEGKDGPALTNGILNTGKKLLGLTERGLTLFAPELDKWIQGNKHLSNSVGSTGNLTKAIDKVQSVLGTLQAFLNTAFSGMDLDALIKARQNGKNVTDVQLAKASLNLINELIGTISSITNNVDTFSKQLNKLGEALGQVKHFGSFGDKLKNLPKLGNLGKGLGALSGVLSAISAALLLANKDADTATKAAAAAELTNKVLGNIGKAITQYLIAQRAAAGLSTTGPVAGLIASVVSLAISPLSFLGIAKQFDRARMLEEYSKRFKKFGYNGDSLLGQFYKNTGIADAAITTINTVLSAIAAGVGAASAGSLVGAPIGLLVSAITSLISGILDASKQAVFEHIANQLADKIKAWENKYGKNYFENGYDARHSAFLEDSLKLFNELREKYKTENILSITQQGWDQRIGELAGITRNGDRIQSGKAYVDYLKKGEELAKHSDKFTKQILDPIKGNIDLSGIKGSTTLTFLNPLLTAGKEERKTRQSGKYEFITELKVKGRTDWKVKGVPNSNGVYDFSNLIQHAVTRDNKVLEARLIANLGAKDDYVFVGSGSTIVNAGDGYDVVDYSKGRTGALTIDGRNATKAGQYKVERDLSGTQVLQETVSKQETKRGKVTDLLEYRNYKLDYYYTNKGFKAHDELNSVEEIIGSTLRDKFYGSKFNDVFHGHDGDDLIYGYDGDDRLYGDNGNDEIHGGQGNDKLYGGAGNDRLFGEYGNNYLDGGEGDDHLEGGNGSDILRGGSGNDKLFGNQGDDLLDGGEGDDQLAGGEGNDIYVYRKEYGHHTITEHSGDKDKLSLANINLKDVSFERNGNDLLLKTNNRTAVTFKGWFSKPNSSAGLDEYQRKLLEYAPEKDRARLKRQFELQRGKVDKSLNNKVEEIIGKDGERITSQDIDNLFDKSGNKKTISPQELAGLIKNKGKSSSLMSSSRSSSMLTQKSGLSNDISRIISATSGFGSSGKALSASPLQTNNNFNSYANSLATTAA

SEQ ID NO: 2 - DNA, 3168 bp, Aggregatibacter actinomycetemcomitans
  source 1..3168: mol_type = genomic DNA
atggcaactacttcactgctaaatacaaaacagcaagctgcacagtttgcaaattcagttgcagatagagctaaggaaaatattgatgctgcaaaagaacaattgcaaaaggcgttagataaattagggaagacaggtaagaaattaactttatatatccctaagaattacaaaaaaggaaatggtcttactgcgcttataaaagcagcacagaagttagggattgaagtatatcatgaagggaaagacggcccggcattaactaatggtattttaaatactgggaaaaaattacttggtcttaccgaacgaggtttaactttatttgctccggaattagataaatggattcaaggtaataaacatttaagtaattctgtgggtagtactggaaatttgacaaaagcgatagataaggttcagagtgttcttggtacgttacaagcgtttttgaacaccgcattttcgggcatggatttagatgccttaattaaagcccgtcaaaatggtaaaaatgtaacagatgtacagctagcaaaagccagtcttaacctgattaatgaattgattggtactatttctagcattacaaataatgtagatactttttctaaacaacttaataagttaggtgaagcactaggacaagtaaaacattttggtagttttggagataaattaaagaatttacctaagttaggtaatcttggaaaaggtttaggtgcattatccggtgtattgtcggctatatcagcggctctattacttgcaaataaagatgctgatactgcaacgaaagcagcggctgcagctgaattgacaaataaagtgctaggtaacatcggtaaagcgatcacacaatacttgattgctcaacgtgctgcagcggggctttctactacgggacctgtcgcagggttaattgcctctgtggtcagcttggcaatcagccctttgtctttcctaggtattgcgaaacaatttgatcgtgcgagaatgcttgaggaatactcgaaacgctttaagaaatttggttataacggcgatagtttacttggtcaattctacaaaaatacagggatcgcagatgctgcgattacaacgattaacactgtattaagtgctattgcagcaggggttggtgcagcctccgccggttctttagttggtgcgccaatcggtttgttagtgagtgcgattaccagcttaatttcaggaattcttgatgcttctaaacaagccgtttttgaacatatcgcgaatcagctcgccgataaaattaaagcatgggagaataagtacggtaagaattactttgaaaatggctatgatgcccgtcattccgccttcttggaagattcactaaaattatttaatgagttacgtgaaaaatataaaaccgaaaatatattatctatcactcaacaaggttgggatcagcgcattggtgaattagcaggtatcactcgtaatggagatcgtattcaaagtggtaaagcttatgtggattatttgaaaaagggtgaggagcttgcaaagcatagcgataaattcactaaacagattttagatccaatcaaaggtaatattgatctttcgggtataaaaggttctaccactctaacttttttaaatccgttgttaaccgcaggtaaggaagaacggaaaacacgtcagtcaggtaaatatgaatttattactgaattaaaagtaaaaggacgtaccgattggaaggtaaaaggtgttcctaattctaatggtgtatatgatttttctaacttaattcaacatgccgttacacgtgataataaagttctagaagcaagattaattgctaatttgggtgctaaagatgattatgtttttgtcggatccggttcaacaatagttaatgctggagacggttatgatgtggtggactatagtaaaggtcgcaccggtgcattaacaatcgacggtcgtaatgctactaaagccggacaatataaggttgaaagagatcttagcggtactcaagtcttgcaggaaaccgtatcaaagcaagaaactaaacgagggaaggttaccgatctacttgaatatcgtaactataaattagattactattatacgaataagggctttaaagctcatgatgaattaaactcagtagaggaaattatcggcagcacactacgtgataaattttatggttctaaatttaatgatgttttccatggtcacgatggcgatgatttgatttatggttatgatggcgatgatcgtttgtatggcgataatgggaatgacgaaattcatggcggccaaggtaatgataagctctatggtggtgccggtaacgataggctctttggtgaatatggcaacaactatcttgacggtggagaaggcgacgaccacttagagggaggcaatggttccgatattctaagaggtggaagtggcaatgataagttgtttggaaaccaaggagatgatttacttgacggtggagaaggcgatgaccaacttgccggtggagaaggaaatgatatttatgtttaccgtaaagaatatgggcaccacactattacggaacatagcggtgataaagataaattatcattagcaaatatcaatctcaaagatgtgtcatttgagcgtaacggcaatgatctactattgaaaacaaataatagaacagcagtaacatttaaaggatggtttagtaaacctaattcatcggcaggattagatgagtatcaaagaaaacttcttgaatacgcacctgaaaaggatcgtgcacgacttaagagacaatttgagttacagcgaggtaaagtcgacaaatcactcaataataaagttgaagaaattatcggtaaagatggggagcggattacttcgcaagacattgataatctttttgataagagtgggaacaaaaagacaatttcacctcaagagcttgccggacttattaagaataaaggtaagtcaagtagccttatgtcttcttctcgttcgtcaagtatgcttacacaaaagtccggtttgtcaaatgatattagtcgtattatttcagcaaccagtggttttggttcatccggtaaagcgttatccgcttcgccattgcagaccaataataactttaactcttacgcaaattcgttagcaactactgcggcc

(19) United States
(12) Patent Application Publication
(10) Pub. No.: US 2025/0319154 A1
(43) Pub. Date: Oct. 16, 2025

(54) COMPOSITIONS AND METHODS FOR TREATING CANCER

(71) Applicant: Rutgers, The State University of New Jersey, New Brunswick, NJ (US)

(72) Inventor: Scott C. Kachlany, Bridgewater, NJ (US)

(73) Assignee: Rutgers, The State University of New Jersey, New Brunswick, NJ (US)

(21) Appl. No.: 18/717,017

(22) PCT Filed: Nov. 30, 2022
(86) PCT No.: PCT/US2022/080670

§ 371 Date: Jun. 6, 2024

Related U.S. Application Data
(60) Provisional application No. 63/286,236, filed on Dec. 6, 2021.

(51) Int. Cl. A61K 38/16; A61K 47/18; C07K 14/285

(52) U.S. Cl. CPC A61K 38/164; A61K 47/18; C07K 14/285

(57) ABSTRACT

This disclosure is based, at least in part, on unexpected discoveries that a novel composition of a leukotoxin (LtxA) polypeptide isolated from Aggregatibatier actin omycetcmcomitans can retain stability and biological activities for an extended period of time even after the composition, is subject to a process of lyophilization, storage, reconstitution, and/or further storage, or under an accelerated condition, and that a particular range of dosage of the LtxA polypeptide and administration regimen can provide high efficacy and low toxicity in treating cancer in a patient in need thereof.

### CROSS-REFERENCE TO RELATED APPLICATIONS

The present application is the U.S. National Phase of International Patent Application Serial No. PCT/US22/80670, filed Nov. 30, 2022, which claims priority under 35 U.S.C. § 119(e) to U.S. Provisional Patent Application No. 63/286,236, filed Dec. 6, 2021. The entire disclosures of the applications noted above are incorporated herein by reference.

### REFERENCE TO AN ELECTRONIC SEQUENCE LISTING

The contents of the electronic sequence listing (Sequence listing 096738.00777.xml; Size: 13,660 bytes; and Date of Creation: Jun. 5, 2024) is herein incorporated by reference in its entirety.

### FIELD OF THE INVENTION

The invention relates to pharmaceutical and biological compositions comprising leukotoxin and methods of use thereof for treating cancer.

### BACKGROUND OF THE INVENTION

Each year, more than 60,500 people die of hematologic malignancies (leukemia, lymphoma, myeloma), with more than 110,000 new cases diagnosed annually in the US alone. Lymphomas are generally classified as Hodgkin's and non-Hodgkin's lymphoma (NHL) which may be T-cell (NK-Natural Killer cells) and or B-cell such as (but not limited to); mantle cell lymphoma (MCL), follicular lymphoma (FL), diffuse large B-cell lymphoma (DLBCL), Burkitt lymphoma etc. Current treatment for these blood cancers includes the use of synthetic compounds that target the cell division process of nearly all cells of the body, not just the cancerous ones. As a result, devastating side effects are all too common. Furthermore, a significant percentage of patients eventually show resistance to many of the drugs, thus rendering treatment largely ineffective and resulting in a high number of patients with relapse, resistance and or refractory diseases. For example, MCL is a deadly and incurable disease, and even with new therapeutic approaches, the mean overall survival rate remains approximately 3-4 years. For FL, the most common indolent NHL, there is no consensus treatment protocol, and the disease is considered incurable. Approximately 30-40% of DLBCL patients still die from this cancer. Most of these deaths result from therapeutic resistance in the cancerous cells when the disease recurs. Thus, there is a great need for novel agents that target B-cell lymphomas. While the drugs currently in use are toxic for cells, they are not highly specific. A new class of therapeutic agents for the treatment of hematologic malignancies, and cancer in general, includes drugs that exhibit specificity for predominantly the cancerous cell type. Examples of targeted therapeutics include Rituximab, which is a monoclonal antibody against B-lymphocytes, and Mylotarg, an antibody-anti-tumor antibiotic fusion directed against cells of myelomonocytic lineage.

The U.S. FDA recently issued an initiative and draft guideline to promote medical research into, and clinical development of experimental therapeutics in combination, to improve clinical outcome, efficacy, and safety profile of cancer drug regimens. Existing standard chemotherapeutic agents are not specific to cancer cells, highly cytotoxic with severe side effects.

Thus, there remains a need to develop new cancer drugs and therapy that target cancer cells, less toxic and effective at treating cancer.

### SUMMARY OF THE INVENTION

This disclosure addresses the need mentioned above in a number of aspects. In one aspect, this disclosure provides a liquid composition for treatment of cancer. The liquid composition comprises: about 0.1 mg/ml to about 0.5 mg/ml of a leukotoxin (LtxA) polypeptide (or protein) isolated from Aggregatibacter (Actinobacillus) actinomycetemcomitans, about 5 mM to about 50 mM Tris, about 100 mM to about 300 mM NaCl, and about 0.05 mM to about 0.5 mM CaCl₂, wherein the liquid composition is formulated to a pH of about 7.0 to about 8.0.

In some embodiments, the liquid composition comprises about 0.3 mg/ml of the LtxA polypeptide. In some embodiments, the liquid composition comprises about 20 mM Tris, about 250 mM NaCl, and about 0.2 mM CaCl₂. In some embodiments, wherein the liquid composition is formulated to a pH of about 7.5.

In some embodiments, the liquid composition is formulated to remain stable for at least 24 hours at 4° C.

In some embodiments, the LtxA polypeptide is isolated from the NJ4500 strain of Aggregatibacter actinomycetemcomitans. In some embodiments, the LtxA polypeptide comprises an amino acid sequence having at least 90% identity to SEQ ID NO: 1 or comprises the amino acid sequence of SEQ ID NO: 1.

In another aspect, this disclosure provides a lyophilized composition prepared from the liquid composition of any one of the preceding claims, wherein the lyophilized composition comprises: about 0.2 mg to about 2 mg of the LtxA polypeptide, about 2 mg to about 8 mg of Tris, about 10 mg to about 50 mg of NaCl, and about 0.01 mg to about 0.5 mg CaCl₂, wherein the lyophilized composition is formulated to have when reconstituted a pH of about 7.0 to 8.0.

In some embodiments, the lyophilized composition comprises: about 0.6 mg of the LtxA polypeptide, about 4.85 mg of Tris, about 29.2 mg of NaCl, and about 0.04 mg of CaCl₂.

In some embodiments, wherein the lyophilized composition is formulated to have a pH of about 7.5 after reconstitution. In some embodiments, the lyophilized composition is reconstituted in sterile water or a saline buffer. In some embodiments, the lyophilized composition is reconstituted as a liquid composition comprising about 0.3 mg/ml of the LtxA polypeptide.

In some embodiments, wherein the lyophilized composition is formulated to remain stable after storage at −20±5° C. up to 24 months. In some embodiments, wherein the lyophilized composition is formulated to remain stable after storage at a temperature lower than −20° C., reconstitution, and then storage for up to 7 days at the temperature lower than −20° C. In some embodiments, wherein the lyophilized composition is formulated to remain stable after storage at a temperature lower than −20° C., reconstitution, and then storage for up to 24 hours at about 4° C.

In another aspect, this disclosure provides a kit comprising the liquid composition or the lyophilized composition, as described herein.

In yet another aspect, this disclosure provides a method for treating cancer in a subject. The method comprises administering to the subject a therapeutically effective amount of the liquid composition described herein, wherein the therapeutically effective amount of the liquid composition is about 1 μg/kg to about 1200 μg/kg based on the weight of the subject or based on a ratio of mass (e.g., in μg) of the liquid composition to a body surface area (e.g., in meter square =M²), such as in μg/M².

In some embodiments, the therapeutically effective amount of the liquid composition is about 1.4 μg/kg based on the weight of the subject. In some embodiments, the therapeutically effective amount of the liquid composition is about 1020 μg/kg based on the weight of the subject or based on a ratio of mass (e.g., in μg) of the liquid composition to a body surface area (e.g., in meter square=M²), such as in μg/M².

In some embodiments, wherein the liquid composition is administered to the subject is formulated as a dosage form selected from modified release, sustained release (depot), controlled release, timed release, delayed release, prolonged release, and/or extended release.

In some embodiments, the liquid composition is administered parenterally by intravenous infusion over a period of 1 to 10 hours. In some embodiments, the liquid composition is administered by intravenous infusion over a period of 3 to 4 hours. In some embodiments, the liquid composition is administered by intravenous infusion over a period of 1 to 2 hours.

In some embodiments, the liquid composition is administered parenterally to the subject in modified release, sustained release (depot), controlled release, timed release, delayed release, prolonged release, and/or extended release.

In some embodiments, the cancer can be any LFA-1-expressing tumors. In some embodiments, the cancer is selected from adrenal gland tumors, biliary cancer, bladder cancer, is brain cancer, breast cancer, carcinoma, central or peripheral nervous system tissue cancer, cervical cancer, colon cancer, endocrine or neuroendocrine cancer or hematopoietic cancer, esophageal cancer, fibroma, gastrointestinal cancer, glioma, head and neck cancer, Li-Fraumeni tumors, liver cancer, lung cancer, leukemia, lymphoma, melanoma, meningioma, multiple neuroendocrine type I and type II tumors, nasopharyngeal cancer, oral cancer, oropharyngeal cancer, osteogenic sarcoma tumors, ovarian cancer, pancreatic cancer, pancreatic islet cell cancer, parathyroid cancer, pheochromocytoma, pituitary tumors, prostate cancer, rectal cancer, renal cancer, respiratory cancer, sarcoma, skin cancer, stomach cancer, testicular cancer, thyroid cancer, tracheal cancer, urogenital cancer, and uterine cancer.

In some embodiments, the cancer is leukemia and any subtype thereof. In some embodiments, the cancer is lymphoma or any subtype thereof.

In some embodiments, lymphoma is selected from Hodgkin lymphoma, and non-Hodgkin lymphoma, including anaplastic large-cell lymphoma, angioimmunoblastic lymphoma, blastic NK-cell lymphoma, Burkitt's lymphoma, Burkitt-like lymphoma (small non-cleaved cell lymphoma), chronic lymphocytic leukemia/small lymphocytic lymphoma, cutaneous T-cell lymphoma, diffuse large B-cell lymphoma, enteropathy-type T-cell lymphoma, follicular lymphoma, hepatosplenic gamma-delta T-cell lymphoma, lymphoblastic lymphoma, mantle cell lymphoma, marginal zone lymphoma, nasal T-cell lymphoma, pediatric lymphoma, peripheral T-cell lymphomas, primary central nervous system lymphoma, transformed lymphomas, treatment-related T-cell lymphomas, and waldenstrom's macroglobulinemia.

In some embodiments, the method comprises further administering to the subject a second agent or therapy. In some embodiments, the second agent comprises an anti-tumor or anti-cancer agent. In some embodiments, the second agent or therapy is administered in sequence before, or after the composition. In some embodiments, the second agent or therapy is administered concurrently with the composition.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features is not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIG. 1 shows the results of qualitative Western Blot for GMP Lot 599-0818-003.

FIG. 2 shows the results of qualitative Western Blot for GMP Lot 599-0718-002.

FIG. 3 shows the results of Western Blot of low dose concentration samples.

FIG. 4 shows the results of characterization of biological activity of low dose concentration samples.

FIG. 5 shows the results of Western Blot of high dose concentration samples.

FIG. 6 shows the results of characterization of biological activity of high dose concentration samples.

### DETAILED DESCRIPTION OF THE INVENTION

This disclosure is based, at least in part, on unexpected discoveries that a novel composition of a leukotoxin (LtxA) polypeptide isolated from Aggregatibacter actinomycetemcomitans can retain stability and biological activities for an extended period of time even after the composition is subject to a process of lyophilization, storage, reconstitution, and/or further storage, or under an accelerated condition, and that a particular range of dosage of the LtxA polypeptide and administration regimen can provide high efficacy and low toxicity in treating cancer in a patient in need thereof.

### Compositions of the LtxA Polypeptide

In one aspect, this disclosure provides a liquid composition for treatment of various cancers. The liquid composition comprises comprising: about 0.1 mg/ml to about 0.5 mg/ml of a leukotoxin (LtxA) polypeptide (or protein) isolated from Aggregatibacter actinomycetemcomitans or a variant/fragment thereof, about 5 mM to about 50 mM Tris, about 100 mM to about 300 mM NaCl, and about 0.05 mM to about 0.5 mM CaCl₂, wherein the liquid composition is formulated to a pH of about 7.0 to about 8.0.

In some embodiments, the liquid composition comprises about 0.3 mg/ml of the LtxA polypeptide. In some embodiments, the liquid composition comprises about 20 mM Tris, about 250 mM NaCl, and about 0.2 mM CaCl₂. In some embodiments, wherein the liquid composition is formulated to a pH of about 7.5. In some embodiments, the liquid composition is formulated to remain stable for at least 24 hours at 4° C.

In some embodiments, the LtxA polypeptide is isolated from the NJ4500 strain of Aggregatibacter actinomycetemcomitans. In some embodiments, the LtxA polypeptide comprises an amino acid sequence having at least 90% identity to SEQ ID NO: 1 or comprises the amino acid sequence of SEQ ID NO: 1.

In another aspect, this disclosure provides a lyophilized composition prepared from the liquid composition of any one of the preceding claims, wherein the lyophilized composition comprises: about 0.2 mg to about 2 mg of the LtxA polypeptide, about 2 mg to about 8 mg of Tris, about 10 mg to about 50 mg of NaCl, and about 0.01 mg to about 0.5 mg CaCl₂, wherein the lyophilized composition is formulated to have when reconstituted a pH of about 7.0 to 8.0.

In some embodiments, the lyophilized composition comprises about 0.6 mg of the LtxA polypeptide, about 4.85 mg of Tris, about 29.2 mg of NaCl, and about 0.04 mg of CaCl₂. In some embodiments, wherein the lyophilized composition is formulated to have a pH of about 7.5 after reconstitution.

In some embodiments, the lyophilized composition is reconstituted in sterile water or a saline buffer. In some embodiments, the lyophilized composition is reconstituted as a liquid composition comprising about 0.3 mg/ml of the LtxA polypeptide.

In some embodiments, wherein the lyophilized composition is formulated to remain stable after storage at −20±5° C. up to 24 months. In some embodiments, wherein the lyophilized composition is formulated to remain stable after storage at a temperature lower than −20° C., reconstitution, and then storage for up to 7 days at the temperature lower than −20C. In some embodiments, wherein the lyophilized composition is formulated to remain stable after storage at a temperature lower than −20° C., reconstitution, and then storage for up to 24 hours at about 4° C.

TABLE 1
Representative Sequences

| SEQ | | |
|---|---|---|
| ID | | OTHER |
| NO | SEQUENCE | INFORMATION |
| 1 | MATTSLLNTKQQAAQFANSVADRAKENIDAAKEQLQKALDK | Aggregatibacter |
| | LGKTGKKLTLYIKNYKKGNGLTALIKAAQKLGIEVYHEGKDG | actinomycetemcomitans |
| | PALTNGILNTGKKLLGLTERGLTLFAPELDKWIQGNKHLSNSV | strain NJ4500 |
| | GSTGNLTKAIDKVQSVLGTLQAFLNTAFSGMDLDALIKARQN | |
| | GKNVTDVQLAKASLNLINELIGTISSITNNVDTFSKQLNKLGE | |
| | ALGQVKHFGSFGDKLKNLPKLGNLGKGLGALSGVLSAISAA | |
| | LLLANKDADTATKAAAAAELTNKVLGNIGKAITQYLIAQRA | |
| | AAGLSTTGPVAGLIASVVSLAISPLSFLGIAKQFDRARMLEEY | |
| | SKRFKKFGYNGDSLLGQFYKNTGIADAAITTINTVLSAIAAG | |
| | VGAASAGSLVGAPIGLLVSAITSLISGILDASKQAVFEHIANQL | |
| | ADKIKAWENKYGKNYFENGYDARHSAFLEDSLKLFNELREK | |
| | YKTENILSITQQGWDQRIGELAGITRNGDRIQSGKAYVDYLK | |
| | KGEELAKHSDKFTKQILDPIKGNIDLSGIKGSTTLTFLNPLLTA | |
| | GKEERKTRQSGKYEFITELKVKGRTDWKVKGVPNSNGVYDF | |
| | SNLIQHAVTRDNKVLEARLIANLGAKDDYVFVGSGSTIVNAG | |
| | DGYDVVDYSKGRTGALTIDGRNATKAGQYKVERDLSGTQVL | |
| | QETVSKQETKRGKVTDLLEYRNYKLDYYYTNKGFKAHDEL | |
| | NSVEEIIGSTLRDKFYGSKFNDVFHGHDGDDLIYGYDGDDRL | |
| | YGDNGNDEIHGGQGNDKLYGGAGNDRLFGEYGNNYLDGGE | |
| | GDDHLEGGNGSDILRGGSGNDKLFGNQGDDLLDGGEGDDQ | |
| | LAGGEGNDIYVYRKEYGHHTITEHSGDKDKLSLANINLKDV | |
| | SFERNGNDLLLKTNNRTAVTFKGWFSKPNSSAGLDEYQRKLL | |
| | EYAPEKDRARLKRQFELQRGKVDKSLNNKVEEIIGKDGERIT | |
| | SQDIDNLFDKSGNKKTISPQELAGLIKNKGKSSSLMSSSRSSS | |
| | MLTQKSGLSNDISRIISATSGFGSSGKALSASPLQTNNNFNSYA | |
| | NSLATTAA | |
| 2 | ATGGCAACTACTTCACTGCTAAATACAAAACAGCAAGCTG | Aggregatibacter |
| | CACAGTTTGCAAATTCAGTTGCAGATAGAGCTAAGGAAAA | actinomycetemcomitans |
| | TATTGATGCTGCAAAAGAACAATTGCAAAAGGCGTTAGATA | strain NJ4500 |
| | AATTAGGGAAGACAGGTAAGAAATTAACTTTATATATCCCT | |
| | AAGAATTACAAAAAAGGAAATGGTCTTACTGCGCTTATAAA | |
| | AGCAGCACAGAAGTTAGGGATTGAAGTATATCATGAAGGG | |
| | AAAGACGGCCCGGCATTAACTAATGGTATTTTAAATACTGG | |
| | GAAAAAATTACTTGGTCTTACCGAACGAGGTTTAACTTTAT | |
| | TTGCTCCGGAATTAGATAAATGGATTCAAGGTAATAAACAT | |
| | TTAAGTAATTCTGTGGGTAGTACTGGAAATTTGACAAAAGC | |
| | GATAGATAAGGTTCAGAGTGTTCTTGGTACGTTACAAGCGT | |
| | TTTTGAACACCGCATTTTCGGGCATGGATTTAGATGCCTTA | |
| | ATTAAAGCCCGTCAAAATGGTAAAAATGTAACAGATGTACA | |
| | GCTAGCAAAAGCCAGTCTTAACCTGATTAATGAATTGATTG | |
| | GTACTATTTCTAGCATTACAAATAATGTAGATACTTTTTCTAA | |
| | ACAACTTAATAAGTTAGGTGAAGCACTAGGACAAGTAAAA | |
| | CATTTTGGTAGTTTTGGAGATAAATTAAAGAATTTACCTAAG | |
| | TTAGGTAATCTTGGAAAAGGTTTAGGTGCATTATCCGGTGT | |
| | ATTGTCGGCTATATCAGCGGCTCTATTACTTGCAAATAAAGA | |
| | TGCTGATACTGCAACGAAAGCAGCGGCTGCAGCTGAATTG | |
| | ACAAATAAAGTGCTAGGTAACATCGGTAAAGCGATCACAC | |
| | AATACTTGATTGCTCAACGTGCTGCAGCGGGGCTTTCTACT | |
| | ACGGGACCTGTCGCAGGGTTAATTGCCTCTGTGGTCAGCTT | |
| | GGCAATCAGCCCTTTGTCTTTCCTAGGTATTGCGAAACAAT | |
| | TTGATCGTGCGAGAATGCTTGAGGAATACTCGAAACGCTTT | |
| | AAGAAATTTGGTTATAACGGCGATAGTTTACTTGGTCAATT | |
| | CTACAAAAATACAGGGATCGCAGATGCTGCGATTACAACG | |
| | ATTAACACTGTATTAAGTGCTATTGCAGCAGGGGTTGGTGC | |
| | AGCCTCCGCCGGTTCTTTAGTTGGTGCGCCAATCGGTTTGT | |
| | TAGTGAGTGCGATTACCAGCTTAATTTCAGGAATTCTTGAT | |
| | GCTTCTAAACAAGCCGTTTTTGAACATATCGCGAATCAGCT | |
| | CGCCGATAAAATTAAAGCATGGGAGAATAAGTACGGTAAG | |
| | AATTACTTTGAAAATGGCTATGATGCCCGTCATTCCGCCTTC | |
| | TTGGAAGATTCACTAAAATTATTTAATGAGTTACGTGAAAA | |
| | ATATAAAACCGAAAATATATTATCTATCACTCAACAAGGTTG | |
| | GGATCAGCGCATTGGTGAATTAGCAGGTATCACTCGTAATG | |
| | GAGATCGTATTCAAAGTGGTAAAGCTTATGTGGATTATTTG | |
| | AAAAAGGGTGAGGAGCTTGCAAAGCATAGCGATAAATTCA | |
| | CTAAACAGATTTTAGATCCAATCAAAGGTAATATTGATCTTT | |
| | CGGGTATaAAAGGTTCTACCACTCTAACTTTTTTAAATCCGT | |
| | TGTTAACCGCAGGTAAGGAAGAACGGAAAACACGTCAGT | |
| | CAGGTAAATATGAATTTATTACTGAATTAAAAGTAAAAGGA | |
| | CGTACCGATTGGAAGGTAAAAGGTGTTCCTAATTCTAATGG | |
| | TGTATATGATTTTTCTAACTTAATTCAACATGCCGTTACACG | |
| | TGATAATAAAGTTCTAGAAGCAAGATTAATTGCTAATTTGG | |
| | GTGCTAAAGATGATTATGTTTTTGTCGGATCCGGTTCAACA | |
| | ATAGTTAATGCTGGAGACGGTTATGATGTGGTGGACTATAG | |
| | TAAAGGTCgCACCGGTGCATTAACAATCGACGGTCGTAATG | |
| | CTACTAAAGCCGGACAATATAAGGTTGAAAGAGATCTTAGC | |
| | GGTACTCAAGTCTTGCAGGAAACCGTATCAAAGCAAGAAA | |
| | CTAAACGAGGGAAGGTTACCGATCTACTTGAATATCGTAAC | |
| | TATAAATTAGATTACTATTATACGAATAAGGGCTTTAAAGCT | |
| | CATGATGAATTAAACTCAGTAGAGGAAATTATCGGCAGCAC | |
| | ACTACGTGATAAATTTTATGGTTCTAAATTTAATGATGTTTTC | |
| | CATGGTCACGATGGCGATGATTTGATTTATGGTTATGATGGC | |
| | GATGATCGTTTGTATGGCGATAATGGGAATGACGAAATTCA | |
| | TGGCGGCCAAGGTAATGATAAGCTCTATGGTGGTGCCGGTA | |
| | ACGATAGGCTCTTTGGTGAATATGGCAACAACTATCTTGAC | |
| | GGTGGAGAAGGCGACGACCACTTAGAGGGAGGCAATGGT | |
| | TCCGATATTCTAAGAGGTGGAAGTGGCAATGATAAGTTGTT | |
| | TGGAAACCAAGGAGATGATTTACTTGACGGTGGAGAAGGC | |
| | GATGACCAACTTGCCGGTGGAGAAGGAAATGATATTTATGT | |
| | TTACCGTAAAGAATATGGGCACCACACTATTACGGAACATA | |
| | GCGGTGATAAAGATAAATTATCATTAGCAAATATCAATCTCA | |
| | AAGATGTGTCATTTGAGCGTAACGGCAATGATCTACTATTG | |
| | AAAACAAATAATAGAACAGCAGTAACATTTAAAGGATGGT | |
| | TTAGTAAACCTAATTCATCGGCAGGATTAGATGAGTATCAA | |
| | AGAAAACTTCTTGAATACGCACCTGAAAAGGATCGTGCAC | |
| | GACTTAAGAGACAATTTGAGTTACAGCGAGGTAAAGTCGA | |
| | CAAATCACTCAATAATAAAGTTGAAGAAATTATCGGTAAAG | |
| | ATGGGGAGCGGATTACTTCGCAAGACATTGATAATCTTTTT | |
| | GATAAGAGTGGGAACAAAAAGACAATTTCACCTCAAGAGC | |
| | TTGCCGGACTTATTAAGAATAAAGGTAAGTCAAGTAGCCTT | |
| | ATGTCTTCTTCTCGTTCGTCAAGTATGCTTACACAAAAGTC | |
| | CGGTTTGTCAAATGATATTAGTCGTATTATTTCAGCAACCAG | |
| | TGGTTTTGGTTCATCCGGTAAAGCGTTATCCGCTTCGCCATT | |
| | GCAGACCAATAATAACTTTAACTCTTACGCAAATTCGTTAG | |
| | CAACTACTGCGGCC | |

Aggregatibacter actinomycetemcomitans is a Gram-negative pathogen that inhabits the oral cavities of humans. A. actinomycetemcomitans is the etiologic agent of localized aggressive periodontitis (LAP), a rapidly progressing and destructive disease of the gingiva and periodontal 5 ligaments. Among its many virulence factors, A. actinomycetemcomitans produces an RTX (repeats in toxin) leukotoxin. A. actinomycetemcomitans leukotoxin is an approximately 115 kDa protein that kills specifically leukocytes of humans and Old-World Primates. Leukotoxin (LtxA) is part of the RTX family that includes E. coli a-hemolysin (H1yA) and Bordetella pertussis adenylate cyclase (CyaA). Leukotoxin may play an important role in A. actinomycetemcomitans pathogenesis by helping the bacterium destroy gingival crevice polymorphonuclear leukocytes (PMNs) and monocytes, resulting in the suppression of local immune defenses.

LtxA binds leukocyte function antigen (LFA-1) on white blood cells (WBCs) and induces cell death via apoptosis or necrosis. It has been found that LtxA preferentially targets WBCs with high levels of activated LFA-1, a characteristic of hematological malignancies such as in leukemias and lymphomas. In many ways, LtxA represents a natural version of an immunotoxin since it is both toxic and highly specific within the same molecule. Advantages of native LtxA over artificially engineered molecules include greater stability, increased specificity, and lower toxicity with minimal side effects.

Since LtxA can identify, target, and kill white blood cells resulting from various types of hematological malignancies such as lymphoma, it is an ideal agent for both the detection and treatment of these conditions. For example, blood from a patient can be analyzed using LtxA-FITC staining. A finding of a large percentage of activated WBCs indicates that the patient should undergo LtxA therapy. The effectiveness of the leukotoxin treatments can be monitored by employing LtxA-FITC reagent that initially diagnosed the disease. As the patient responds positively to treatment, the number of WBCs with upregulated activated surface LFA-1 should be seen to decrease. Further, because of LtxA's highly specificity, and targeting ability, few side effects are expected. LtxA is able to kill many leukemia and lymphoma cell lines, and preclinical studies have shown that it may be an effective targeted therapy for treating hematological malignancies. In non-human primates, it was found that a single LtxA treatment depleted leukocyte counts after only 12 hours (quick onset of activity). Importantly, high doses administered to mice were found to be non-toxic.

While many LtxA preparations can be used, highly purified LtxA is preferred. Examples include LtxA polypeptide purified from Aggregatibacter actinomycetemcomitans (SEQ ID NO: 1) and other variants having substantially the same biological activity as that having the sequence of SEQ ID NO: 1. It was discovered that Aggregatibacter actinomycetemcomitans secreted active LtxA into culture supernatants (Kachlany, S. C., et al. 2000. Infect Immun 68:6094-100), and an efficient method for its purification was described in Kachlany, S. C., et al. 2002. Protein Expr Purif 25:465-71. This method can therefore be used to prepare isolated or purified LtxA polypeptide.

In one example, a purification procedure of the toxin involves: (a) inoculating a single colony of Aggregatibacter actinomycetemcomitans into a fresh broth and growing cultures; (b) adding the growing cultures to fresh broth, adding glass beads, and incubating; (c) centrifuging the incubated culture, forming a pellet and a supernatant; (d) filtering the supernatant through a membrane to provide a filtered supernatant; (e) mixing (NH₄)₂SO₄ and the filtered supernatant together to form a mixture; (f) centrifuging the mixture to form a mixture pellet; (g) resuspending the mixture pellet in a buffer to form a protein resuspension; (h) passing the protein resuspension through a column; and (i) collecting the protein eluting off the column. See also PCT/US2006/45258 (WO 2007/062150) and US Application 20090075883 (U.S. Ser. No. 12/154,843). The contents of these two documents are incorporated herein by reference in their entireties.

Various bacterial LtxA or variants thereof can be used in this disclosure. For example, forms of LtxA include the JP2 form (isolated from the JP2 strain of Aggregatibacter actinomycetemcomitans) and the NJ4500 form (isolated from the NJ4500 strain of Aggregatibacter actinomycetemcomitans). The NJ4500 strain of Aggregatibacter actinomycetemcomitans was deposited with the American Type Culture Collection (ATCC), University Boulevard, Manassas, Va., 20110-2209, USA, as Accession Number PTA-11721 on Mar. 2, 2011.

The terms “polypeptide,” “oligopeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified, for example, by disulfide bond formation, glycosylation, lipidation, acetylation, phosphorylation, pegylation, or any other manipulation, such as conjugation with a labeling component. As used herein, the term “amino acid” includes natural and/or unnatural or synthetic amino acids, including glycine and both the D or L optical isomers, and amino acid analogs and peptidomimetics.

An “isolated polypeptide” refers to a polypeptide that has been separated from other proteins, lipids, and nucleic acids with which it is naturally associated. The polypeptide can constitute at least 10% (i.e., any percentage between 10% and 100%, e.g., 20%, 30%, 40%, 50%, 60%, 70%, 80%, 85%, 90%, 95%, and 99%) by dry weight of the purified preparation. Purity can be measured by any appropriate standard method, for example, by column chromatography, polyacrylamide gel electrophoresis, or HPLC analysis. An isolated polypeptide of the invention can be purified from a natural source, produced by recombinant DNA techniques, or by chemical methods. A functional equivalent of LtxA refers to a polypeptide derivative of the LtxA polypeptide, e.g., a protein having one or more, point-mutation(s), insertions, deletions, truncations, a fusion protein, or a combination thereof. It retains substantially the activity of the LtxA polypeptide, i.e., the ability to target and kill WBCs that express the activated conformation of LFA-1 on their surface while having little or no toxic effect on other cells or organs in the body. The isolated polypeptide can contain SEQ ID NO: 1 or a functional fragment of SEQ ID NO: 1. In general, the functional equivalent is at least 75% (e.g., any number between 75% and 100%, inclusive, e.g., 70%, 80%, 85%, 90%, 95%, and 99%) identical to SEQ ID NO: 1.

All naturally occurring LtxA, genetically engineered LtxA, and chemically synthesized LtxA can be used to practice the invention disclosed herein. LtxA obtained by recombinant DNA technology may have the same amino acid sequence as naturally an occurring LtxA (SEQ ID NO: 1) or a functionally equivalent thereof. The term “LtxA” also covers chemically modified LtxA. Examples of chemically modified LtxA include LtxA subjected to a conformational change(s), addition, and or deletion of a sugar chain, and LtxA, to which a compound such as polyethylene glycol has been bound. Once purified and tested by standard methods known in the art, LtxA can be included in a pharmaceutical and/or biological composition.

A LtxA polypeptide, as described herein, can be obtained as a naturally occurring polypeptide or a recombinant polypeptide. To prepare a recombinant polypeptide, a nucleic acid encoding it (e.g., SEQ ID NO: 2) can be linked to another nucleic acid encoding a fusion partner, e.g., glutathione-s-transferase (GST), 6×His epitope tag, or M13 Gene 3 protein. The resultant fusion nucleic acid expresses in suitable host cells a fusion protein that can be isolated by methods known in the art. The isolated fusion protein can be further treated, e.g., by enzymatic digestion, to remove the fusion partner and obtain the recombinant polypeptide of this disclosure.

Also within the scope of this disclosure are the variants of the LtxA protein or polypeptide, as described above. As used herein, the term “variant” refers to a first molecule that is related to a second molecule (also termed a “parent” molecule). The variant molecule can be derived from, isolated from, based on or homologous to the parent molecule. A “functional variant” of a protein as used herein refers to a variant of such protein that retains at least partially the activity of that protein. Functional variants may include mutants (which may be insertion, deletion, or replacement mutants), including polymorphs, etc. Also included within functional variants are fusion products of such protein with another, usually unrelated, nucleic acid, protein, polypeptide, or peptide. Functional variants may be naturally occurring or may be man-made.

A peptide or polypeptide “fragment” as used herein refers to a less than full-length peptide, polypeptide, oligopeptide, or protein. For example, a peptide, oligopeptide, or polypeptide fragment can have at least about 3, at least about 4, at least about 5, at least about 10, at least about 20, at least about 30, at least about 40 amino acids in length, or single unit lengths thereof. For example, fragment may be 6, 7, 8, 9, 10, 11, 12, 13, 14, 15, 16, 17, or more amino acids in length. There is no upper limit to the size of a peptide fragment. However, in some embodiments, peptide fragments can be less than about 500 amino acids, less than about 400 amino acids, less than about 300 amino acids or less than about 250 amino acids in length.

The amino acid composition of the LtxA polypeptide described herein may vary without disrupting the ability of the polypeptide to target and kill WBCs. For example, it can contain one or more conservative amino acid substitutions. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in SEQ ID NO: 1 is preferably replaced with another amino acid residue from the same side chain family. Alternatively, mutations can be introduced randomly along all or part of SEQ ID NO: 1, such as by saturation mutagenesis, and the resultant mutants can be screened for the ability to improve skin condition to identify mutants that retain the activity as described below in the examples.

As used herein, the percent homology between two amino acid sequences is equivalent to the percent identity between the two sequences. The percent identity between the two sequences is a function of the number of identical positions shared by the sequences (i.e., % homology=#of identical positions/total #of positions×100), considering the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program, using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm, which has been incorporated into the GAP program in the GCG software package (available at www.gcg.com), using either a Blossum62 matrix or a PAM250 matrix, and a gap weight of 16, 14, 12, 10, 8, 6, or 4 and a length weight of 1, 2, 3, 4, 5, or 6.

The term “homolog” or “homologous,” when used in reference to a polypeptide, refers to a high degree of sequence identity between two polypeptides, or to a high degree of similarity between the three-dimensional structure or to a high degree of similarity between the active site and the mechanism of action. In some embodiments, a homolog has a greater than 60% sequence identity, and more preferably greater than 75% sequence identity, and still more preferably greater than 90% sequence identity, with a reference sequence. The term “substantial identity,” as applied to polypeptides, means that two peptide sequences, when optimally aligned, such as by the programs GAP or BESTFIT using default gap weights, share at least 75% sequence identity.

Also within the scope of this disclosure are the variants, mutants, and homologs with significant identity to the disclosed LtxA polypeptides. For example, such variants and homologs may have sequences with at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the sequences of LtxA polypeptides described herein.

In some embodiments, the detectable tag can be conjugated or linked to the N- and/or C-terminus of a LtxA polypeptide or a variant thereof. The detectable tag and the affinity tag may also be separated by one or more amino acids. In some embodiments, the detectable tag can be conjugated or linked to the variant via a cleavable element. In the context of the present invention, the term “cleavable element” relates to peptide sequences that are susceptible to cleavage by chemical agents or enzyme means, such as proteases. Proteases may be sequence-specific (e.g., thrombin) or may have limited sequence specificity (e.g., trypsin). Cleavable elements I and II may also be included in the amino acid sequence of a detection tag or polypeptide, particularly where the last amino acid of the detection tag or polypeptide is K or R.

As used herein, the term “conjugate” or “conjugation” or “linked” as used herein refers to the attachment of two or more entities to form one entity. A conjugate encompasses both peptide-small molecule conjugates as well as peptide-protein/peptide conjugates.

The term “fusion polypeptide” or “fusion protein” or “fusion oligopeptide” means a protein created by joining two or more polypeptide sequences together. The fusion polypeptides encompassed in this invention include translation products of a chimeric gene construct that joins the nucleic acid sequences encoding a first polypeptide with the nucleic acid sequence encoding a second polypeptide to form a single open reading frame. In other words, a “fusion polypeptide” or “fusion protein” is a recombinant protein of two or more proteins that are joined by a peptide bond or via several peptides. The fusion protein may also comprise a peptide linker between the two domains.

The term “linker” refers to any means, entity, or moiety used to join two or more entities. A linker can be a covalent linker or a non-covalent linker. Examples of covalent linkers include covalent bonds, or a linker moiety covalently attached to one or more of the proteins or domains to be linked. The linker can also be a non-covalent bond, e.g., an organometallic bond through a metal center such as a platinum atom. For covalent linkages, various functionalities can be used, such as amide groups, including carbonic acid derivatives, ethers, esters, including organic and inorganic esters, amino, urethane, urea, and the like. To provide for linking, the domains can be modified by oxidation, hydroxylation, substitution, reduction etc., to provide a site for coupling. Methods for conjugation are well known by persons skilled in the art and are encompassed for use in the present invention. Linker moieties include, but are not limited to, chemical linker moieties, or for example, a peptide linker moiety (a linker sequence).

In some embodiments, the linker can be a peptide linker or a non-peptide linker. Examples of the peptide linker may include, without limitation, [S(G)n]m or [S(G)n]mS, where n may be an integer between 1 and 20, and m may be an integer between 1 and 10.

As used herein, the term “stability” refers to protein stability and/or biological activity (e.g., the ability of the polypeptide to target and kill WBCs). In some embodiments, the term “stability,” as used herein, refers to the ability of a molecule to maintain a folded state under a condition (e.g., storage condition) such that it retains at least one of its normal functional activities, for example, binding to a target molecule or targeting and killing WBCs. In some embodiments, a polypeptide may have a reduced stability when denaturation, aggregation, or oligomerization occurs during storage, lyophilization, or freezing/refreezing. Measurement of protein stability and protein lability can be viewed as the same or different aspects of protein integrity. Proteins are sensitive or “labile” to denaturation caused by heat, by ultraviolet or ionizing radiation, changes in the ambient osmolarity and pH if in liquid solution, mechanical shear force imposed by small pore-size filtration, ultraviolet radiation, ionizing radiation, such as by gamma irradiation, chemical or heat dehydration, or any other action or force that may cause protein structure disruption. The stability of the molecule can be determined using standard methods. For example, the stability of a molecule can be determined by measuring the thermal melting (“TM”) temperature, the temperature in degree Celsius (° C.) at which ½ of the molecules become unfolded, using standard methods.

Typically, the higher the TM, the more stable the molecule. In addition to heat, the chemical environment also changes the ability of the protein to maintain a particular three-dimensional structure. In some embodiments, protein stability may be characterized by Western Blot, Mass spectrometry (MS), high-performance liquid chromatography (HPLC), immunoassays (e.g., ELISA), or an ATP-based Cell Viability Assay.

Any suitable agent may be used to adjust the pH of the composition. Typical agents which may be used to adjust the pH may be one or more of the following: NaOH, NH₄OH, hydrochloric acid, acetic acid, sulphuric acid, EDTA, Tris buffer, etc. In one example, the pH of the sample is adjusted using a base such as sodium hydroxide or an acid such as hydrochloric acid. The pharmaceutical compositions of this disclosure may be formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN), DNA conjugates, anhydrous absorption pastes, oil-in-water, and water-in-oil emulsions, emulsions carbowax (polyethylene glycols of various molecular weights), semi-solid gels, and semi-solid mixtures containing carbowax. See also Powell et al. PDA (1998) J Pharm Sci Technol 52:238-311.

As used herein, the term “composition” or “pharmaceutical composition” refers to a mixture of at least one component useful within this disclosure with other components, such as carriers, stabilizers, diluents, dispersing agents, suspending agents, thickening agents, and/or excipients. The pharmaceutical composition facilitates administration of one or more components of the invention to an organism.

The pharmaceutical compositions generally comprise substantially purified LtxA and a pharmaceutically acceptable carrier in a form suitable for administration to a subject. Pharmaceutically acceptable carriers are determined in part by the specific composition being administered, as well as by the particular method used to administer the composition. The pharmaceutical compositions are generally formulated as sterile, substantially isotonic and in full compliance with all Good Manufacturing Practice (GMP) regulations of the U.S. Food and Drug Administration.

The terms “pharmaceutically acceptable,” “physiologically tolerable,” as referred to compositions, carriers, diluents, and reagents, are used interchangeably and include materials are capable of administration to or upon a subject without the production of undesirable physiological effects to the degree that would prohibit administration of the composition. For example, “pharmaceutically-acceptable excipient” includes an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous. Examples of such carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with LtxA, use thereof in the compositions is contemplated. Supplementary active compounds can also be incorporated into the compositions.

A pharmaceutical composition is formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water-soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate-buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. In many cases, it will be preferable to include isotonic compounds, e.g., sugars, polyalcohols such as mannitol, sorbitol, sodium chloride in the composition. Prolonged absorption of the injectable compositions can be brought about by including in the composition a compound which delays absorption, e.g., aluminum monostearate and gelatin.

Sterile injectable solutions can be prepared by incorporating LtxA in the required amount in an appropriate solvent with one or a combination of ingredients enumerated above, as required. Generally, dispersions are prepared by incorporating LtxA into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof. LtxA can be administered in the form of a depot injection or implant preparation, which can be formulated in such a manner as to permit a sustained or pulsatile release of the active ingredient.

Also within the scope of this disclosure is a kit comprising the liquid composition or the lyophilized composition, as described herein, e.g., for treating or inhibiting the growth of a tumor of a patient. In some embodiments, the kit also includes a container that contains the composition and optionally informational material. The informational material can be descriptive, instructional, marketing or other material that relates to the methods described herein and/or the use of the agents is for therapeutic benefit. In an embodiment, the kit also includes an additional therapeutic agent, as described herein. For example, the kit includes a first container that contains the composition and a second container for the additional therapeutic agent.

The informational material of the kits is not limited in its form. In some embodiments, the informational material can include information about production of the composition, concentration, date of expiration, batch or production site information, and so forth. In one embodiment, the informational material relates to methods of administering the composition, e.g., in a suitable dose, dosage form, or mode of administration (e.g., a dose, dosage form, or mode of administration described herein), to treat a subject in need thereof. In one embodiment, the instructions provide a dosing regimen, dosing schedule, and/or route of administration of the composition or the additional therapeutic agent. The information can be provided in a variety of formats, including printed text, computer-readable material, video recording, or audio recording, or information that contains a link or address to substantive material.

The kit can include one or more containers for the composition. In some embodiments, the kit contains separate containers, dividers, or compartments for the composition and informational material. For example, the composition can be contained in a bottle or vial, and the informational material can be contained in a plastic sleeve or packet. In other embodiments, the separate elements of the kit are contained within a single, undivided container. For example, the composition is contained in a bottle or vial that has attached thereto the informational material in the form of a label. In some embodiments, the kit includes a plurality (e.g., a pack) of individual containers, each containing one or more unit dosage forms (e.g., a dosage form described herein) of the agents.

The kit optionally includes a device suitable for administration of the composition or other suitable delivery device. The device can be provided pre-loaded with one or both of the agents or can be empty, but suitable for loading. Such a kit may optionally contain a syringe to allow for injection of the antibody contained within the kit into an animal, such as a human.

### Methods of Treating Cancer

In yet another aspect, the present disclosure includes methods for treating, delaying, or inhibiting the growth of a tumor. In some embodiments, the present disclosure includes methods to promote tumor growth inhibition, cancer regression, and/or induction of cancer apoptosis. In some embodiments, the present disclosure includes methods to reduce tumor cell load or to reduce tumor burden. In some embodiments, the present disclosure includes methods to prevent tumor recurrence, resistance, relapse, and/or refractory.

In one aspect, this disclosure provides a method for treating cancer in a subject. The method comprises administering to the subject a therapeutically effective amount of the liquid composition described herein, wherein the therapeutically effective amount of the liquid composition is about 1 μg/kg to about 1200 μg/kg (e.g., 1.4, 2, 4, 6, 8, 10, 20, 40, 60, 80, 100, 200, 300, 400, 500, 600, 700, 800, 900, 1000, 1100, 1200 μg/kg) based on the weight of the subject.

In some embodiments, the therapeutically effective amount of the liquid composition is about 1.4 μg/kg based on the weight of the subject. In some embodiments, the therapeutically effective amount of the liquid composition is about 1020 μg/kg based on the weight of the subject.

The dose of the pharmaceutical composition of the present invention is determined according to the age, body weight, skin surface area, general health condition, sex, diet, administration time, administration method, clearance rate, and or the level of disease for which patients are undergoing treatments at that time, or further in consideration of other factors. While the daily dose of the compound of the present invention varies depending on the condition and body weight of patient, the kind of the compound, administration route and the like, it is parenterally administered at, for example, 0.001 to 100 mg/patient/day by subcutaneous, intravenous, intramuscular, transdermal, transocular, transpulmonary bronchial, or transnasal administration. Variations in the needed dosage are to be expected in view of the variety of compounds available and the different efficiencies of various routes of administration. Variations in these dosage levels can be adjusted using standard empirical routines for optimization as is well understood in the art. Encapsulation of the compound in a suitable delivery vehicle (e.g., polymeric microparticles or implantable devices) may increase the efficiency of delivery.

Oral dosage forms may include capsules, tablets, emulsions and aqueous suspensions, dispersions, and solutions. In the case of tablets, commonly used carriers include, but are not limited to, lactose and corn starch. Lubricating agents, such as, but not limited to, magnesium stearate, also are typically added. For oral administration in a capsule form, useful diluents include, but are not limited to, lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added. In some embodiments, an oral dosage range is from about 1.0 to about 100 mg/kg body weight administered orally in single or divided doses, including from about 1.0 to about 50 mg/kg body weight, from about 1.0 to about 25 mg/kg body weight, from about 1.0 to about 10 mg/kg body weight (assuming an average body weight of approximately 70 kg; values adjusted accordingly for persons weighing more or less than average). For oral administration, the compositions are, for example, provided in the form of a tablet containing from about 50 to about 1000 mg of the active ingredient, particularly about 75 mg, about 100 mg, about 200 mg, about 400 mg, about 500 mg, about 600 mg, about 750 mg, or about 1000 mg of the active ingredient for the symptomatic adjustment of the dosage to the subject being treated.

In some embodiments, the pharmaceutical composition as described herein is administered to a subject by various methods that may include continuous or intermittent administration, depending on the nature of the cancer. The pharmaceutical compositions may be administered by routes independently selected from the group consisting of oral administration, intravenous administration, intraarterial administration, intramuscular administration, intracolonic administration, intracranial administration, intrathecal administration, intraventricular administration, intraurethral administration, intravaginal administration, subcutaneous administration, intraocular administration, intranasal administration, and any combinations thereof. Accordingly, the pharmaceutically effective compositions may also include pharmaceutically acceptable additives, carriers or excipients. Such pharmaceutical compositions may also include the active ingredients formulated together with one or more non-toxic, pharmaceutically acceptable carriers specially formulated for oral administration in solid or liquid form, for parenteral injection or for rectal administration according to standard methods known in the art.

The term “parenteral” administration refers to modes of administration which include intravenous, intramuscular, intraperitoneal, intracisternal, intratarsal, subcutaneous, and intraarticular injection and infusion. Injectable mixtures are known in the art and comprise pharmaceutically acceptable sterile aqueous or nonaqueous solutions, dispersions, suspensions, or emulsions as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol and the like), vegetable oils (such as olive oil), injectable organic esters (such as ethyl oleate) and suitable mixtures thereof.

In some embodiments, the liquid composition is administered to the subject intravenously, subcutaneously, or intraperitoneally.

In some embodiments, the liquid composition is administered by intravenous infusion. In some embodiments, the liquid composition is administered by intravenous infusion over a period of 1 to 10 hours (e.g., 1, 2, 3, 4, 5, 6, 7, 8, 9, 10 hours). In some embodiments, the liquid composition is administered by intravenous infusion over a period of 3 to 4 hours (+/−15 minutes).

In some embodiments, the liquid composition is administered by intravenous infusion over a period of 1 to 2 hours (+/−15 minutes).

In some embodiments, the liquid composition is administered to the subject in sustained release, in controlled release, in delayed release.

Various delivery systems are known and can be used to administer the pharmaceutical composition of the present disclosure, e.g., encapsulation in liposomes, microparticles, microcapsules, recombinant cells capable of expressing the mutant viruses, receptor-mediated endocytosis (see, e.g., Wu et al., 1987, J. Biol. Chem. 262: 4429-4432). Methods of administration include, but are not limited to, intravesical, intradermal, intramuscular, intratumoral, intraperitoneal, intravenous, subcutaneous, intranasal, epidural, and oral routes. The composition may be administered by any convenient route, for example, by infusion or bolus injection, by absorption through epithelial or mucocutaneous linings (e.g., oral mucosa, rectal and intestinal mucosa, etc.) and may be administered together with other biologically active agents.

As used herein, the term “subject” may be interchangeably used with the term “patient.” The expression “a subject in need thereof” means a human or non-human mammal that exhibits one or more symptoms or indications of cancer and/or who has been diagnosed with cancer. In some embodiments, a human subject may be diagnosed with a primary or a metastatic tumor and/or with one or more symptoms or indications including, but not limited to, enlarged lymph node(s), swollen abdomen, chest pain/pressure, unexplained weight loss, fever, night sweats, persistent fatigue, loss of appetite, enlargement of spleen, itching. The expression includes patients who have received one or more cycles of chemotherapy with toxic side effects. In some embodiments, the expression “a subject in need thereof” includes patients with cancer that has been treated but which has subsequently relapsed or metastasized. For example, patients that may have received treatment with one or more anti-cancer agents leading to tumor regression; however, subsequently have relapsed with cancer resistant to the one or more anti-cancer agents (e.g., chemotherapy-resistant cancer) are treated with the methods of the present disclosure.

As used herein, the terms “treating,” “treat,” or the like mean to alleviate or reduce the severity of at least one symptom or indication, to eliminate the causation of symptoms either on a temporary or permanent basis, to delay or inhibit tumor growth, to reduce tumor cell load or tumor burden, to promote tumor regression, to cause tumor shrinkage, necrosis and/or disappearance, to prevent tumor recurrence, to prevent or inhibit metastasis, to inhibit metastatic tumor growth, to eliminate the need for radiation or surgery, and/or to increase duration of survival of the subject.

The term “effective amount,” “effective dose,” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A “prophylactically effective amount” or a “prophylactically effective dosage” of a drug is an amount of the drug that, when administered alone or in combination with another therapeutic agent to a subject at risk of developing a disease or of suffering a recurrence of disease, inhibits the development or recurrence of the disease. The ability of a therapeutic or prophylactic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

In many embodiments, the terms “tumor,” “lesion,” “tumor lesion,” “cancer,” and “malignancy” are used interchangeably and refer to one or more cancerous growths. In some embodiments, the cancer can be any LFA-1-expressing tumors. In some embodiments, the cancer is selected from adrenal gland tumors, biliary cancer, bladder cancer, brain cancer, breast cancer, carcinoma, central or peripheral nervous system tissue cancer, cervical cancer, colon cancer, endocrine or neuroendocrine cancer or hematopoietic cancer, esophageal cancer, fibroma, gastrointestinal cancer, glioma, head and neck cancer, Li-Fraumeni tumors, liver cancer, lung cancer, leukemia, lymphoma, melanoma, meningioma, multiple neuroendocrine type I and type II tumors, nasopharyngeal cancer, oral cancer, oropharyngeal cancer, osteogenic sarcoma tumors, ovarian cancer, pancreatic cancer, pancreatic islet cell cancer, parathyroid cancer, pheochromocytoma, pituitary tumors, prostate cancer, rectal cancer, renal cancer, respiratory cancer, sarcoma, skin cancer, stomach cancer, testicular cancer, thyroid cancer, tracheal cancer, urogenital cancer, and uterine cancer.

In some embodiments, the cancer is leukemia or a subtype thereof. For example, leukemia can be an acute or chronic leukemia of a lymphocytic or myelogenous origin, such as, but not limited to: Acute lymphoblastic leukemia (ALL); Acute myelogenous leukemia (AML); Chronic lymphocytic leukemia (CLL); Chronic myelogenous leukemia (CML); juvenile myelomonocytic leukemia (JMML); hairy cell leukemia (HCL); acute promyelocytic leukemia (a subtype of AML); large granular lymphocytic leukemia; or Adult T- cell chronic leukemia. In one embodiment, the patient suffers from an acute myelogenous leukemia, for example an undifferentiated AML (MO); myeloblastic leukemia (M1; with/without minimal cell maturation); myeloblastic leukemia (M2; with cell maturation); promyelocytic leukemia (M3 or M3 variant [M3V]); myelomonocytic leukemia (M4 or M4 variant with eosinophilia [M4E]); monocytic leukemia (M5); erythroleukemia (M6); or megakaryoblastic leukemia (M7).

The lymphoma may include lymphoma cells expressing activated LFA-1, and the leukotoxin binds to the activated LFA-1 on the lymphoma cells and destroys the lymphoma cells by apoptosis or necrosis, thereby treating the lymphoma. In some embodiments, lymphoma is selected from Hodgkin lymphoma, and non-Hodgkin lymphoma, including anaplastic large-cell lymphoma, angioimmunoblastic lymphoma, blastic NK-cell lymphoma, burkitt's lymphoma, burkitt-like lymphoma (small non-cleaved cell lymphoma), chronic lymphocytic leukemia/small lymphocytic lymphoma, cutaneous T-cell lymphoma, diffuse large B-cell lymphoma, enteropathy-type T-cell lymphoma, follicular lymphoma, hepatosplenic gamma-delta T-cell lymphoma, lymphoblastic lymphoma, mantle cell lymphoma, marginal zone lymphoma, nasal T-cell lymphoma, pediatric lymphoma, peripheral T-cell lymphomas, primary central nervous system lymphoma, transformed lymphomas, treatment-related T-cell lymphomas, and waldenstrom's macroglobulinemia.

In some embodiments, the methods of the present disclosure further include administering to a subject a second agent or therapy. Anti-tumor therapies include, but are not limited to, conventional anti-tumor therapies such as chemotherapy, radiation, surgery, or as elsewhere described herein. The second agent or therapy may be administered for increasing anti-tumor efficacy, for reducing toxic effects of one or more therapies and/or for reducing the dosage of one or more therapies.

In some embodiments, the second agent may include a chemotherapeutic pharmaceutical. Non-limiting examples of chemotherapeutic pharmaceuticals include idarubicin, cytarabine, etosposide, daunorubicin, mitoxantrone, and melphalan. Other common chemotherapeutic agents for the treatment of leukemia and lymphoma include Chlorambucil, Fludarabine phosphate, Cytarabine, and Daunorubicin hydrochloride. These drugs share the common property of being highly toxic to humans, affecting many different tissue and organ systems of the body. Bone marrow suppression, severe neurologic effects, infertility, pulmonary, and gastrointestinal effects are some of the adverse effects exhibited by these drugs. Many of these drugs act by inhibiting DNA synthesis, a process that all dividing cells carry out. Most cells of the body are targeted by these non-specific pharmaceuticals. Any suitable pharmaceutical agent may be used in conjunction with LtxA, and the combination of a pharmaceutical agent with leukotoxin is intended to reduce the dose of the pharmaceutical necessary to achieve effective results in patients.

In some embodiments, a second agent or therapy may include one or more of: radiation, surgery, a cancer vaccine, imiquimod, an anti-viral agent (e.g., cidofovir), photodynamic therapy, any of immune checkpoint molecules; a CTLA4, PD-1/PD-L1 pathway inhibitor (e.g., an anti-PD-1 antibody, an anti-PD-L1 antibody), a lymphocyte activation gene 3 (LAG3) inhibitor (e.g., an anti-LAG3 antibody, a glucocorticoid-induced tumor necrosis factor receptor (GITR) agonist (e.g., an anti-GITR antibody), a T-cell immunoglobulin and mucin containing -3 (TIM3) inhibitor, a Band T-lymphocyte attenuator (BTLA) inhibitor, a T-cell immunoreceptor with Ig and ITIM domains (TIGIT) inhibitor, a CD38 inhibitor, a CD47 inhibitor, an indoleamine-2,3-dioxygenase (IDO) inhibitor, a CD28 activator, a vascular endothelial growth factor (VEGF) antagonist (e.g., a “VEGF-Trap” such as aflibercept, or an anti-VEGF antibody or antigen-binding fragment thereof (e.g., bevacizumab, or ranibizumab) or a small molecule kinase inhibitor of VEGF receptor (e.g., sunitinib, sorafenib, or pazopanib)), an angiopoietin-2 (Ang2) inhibitor, a transforming growth factor beta (TGFβ) inhibitor, an epidermal growth factor receptor (EGFR) inhibitor, an antibody to a tumor-specific antigen (e.g., CA9, CA125, melanoma-associated antigen 3 (MAGE3), carcinoembryonic antigen (CEA), vimentin, tumor-M2-PK, prostate-specific antigen (PSA), mucin-1, MART-1, and CA19-9), a Bacillus Calmette-Guerin (BCG) therapy, a vaccine (e.g., Bacillus Calmette-Guerin (BCG)), granulocyte-macrophage colony-stimulating factor (GM-CSF), a second oncolytic virus, a cytotoxin, a chemotherapeutic agent (e.g., pemetrexed, dacarbazine, temozolomide, cyclophosphamide, docetaxel, doxorubicin, daunorubicin, cisplatin, carboplatin, gemcitabine, methotrexate, mitoxantrone, oxaliplatin, paclitaxel, topotecan, irinotecan, vinorelbine, and vincristine), an IL-6R inhibitor, an IL-4R inhibitor, an IL-10 inhibitor, a cytokine such as IL-2, IL-7, IL-12, and IL-21, an antibody drug conjugate, an anti-inflammatory drug such as a corticosteroid, a non-steroidal anti-inflammatory drug (NSAID), cryotherapy, anti-HPV therapy, laser therapy, electrosurgical excision of cells with HPV, and combinations thereof.

In some embodiments, the methods further comprise administering a second agent, such as an anti-cancer drug. As used herein, “anti-cancer drug” means any agent useful to treat cancer including, but not limited to, cytotoxins and agents such as antimetabolites, alkylating agents, anthracyclines, antibiotics, antimitotic agents, procarbazine, hydroxyurea, asparaginase, corticosteroids, mitotane (O, P′-(DDD)), biologics (e.g., antibodies and interferons) and radioactive agents. As used herein, “a cytotoxin or cytotoxic agent” also refers to a chemotherapeutic agent and means any agent that is detrimental to cells. Examples include TAXOL (paclitaxel), temozolomide, cytochalasin B, gramicidin D, ethidium bromide, emetine, cisplatin, mitomycin, etoposide, teniposide, vincristine, vinblastine, colchicine, doxorubicin, daunorubicin, dihydroxy anthracene dione, mitoxantrone, mithramycin, actinomycin D, 1-dihydrotestosterone, glucocorticoids, procaine, tetracaine, lidocaine, propranolol, and puromycin and analogs or homologs thereof.

In some embodiments, the second agent or therapy is administered before or after the composition. In some embodiments, the second agent or therapy is administered concurrently with the composition.

As used herein, the term “in combination with” also includes sequential or concomitant administration of the LtxA polypeptide and a second agent (e.g., therapeutic agent) or therapy. For example, when administered “before” a second agent or therapy, one or more doses of the LtxA polypeptide may be administered more than about 12 weeks, about 11 weeks, about 10 weeks, is about 9 weeks, about 8 weeks, about 7 weeks, about 6 weeks, about 5 weeks, about 4 weeks, about 3 weeks, about 2 weeks, about 150 hours, about 150 hours, about 100 hours, about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes or about 10 minutes prior to the administration of one or more doses of the LtxA polypeptide.

When administered “after” a second agent or therapy, the LtxA polypeptide may be administered about 12 weeks, about 11 weeks, about 10 weeks, about 9 weeks, about 8 weeks, about 7 weeks, about 6 weeks, about 5 weeks, about 4 weeks, about 3 weeks, about 2 weeks, about 150 hours, about 150 hours, about 100 hours, about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes or about 10 minutes after the administration of the IL-15 polypeptide.

Administration “concurrent” with the second agent or therapy means that the LtxA polypeptide is administered to the subject in a separate dosage form within less than 10 minutes (before, after, or at the same time) of administration of a second agent or therapy or administered to the subject as a single combined dosage formulation comprising both the LtxA polypeptide and a second agent or therapy.

In some embodiments, the treatment produces a therapeutic effect selected from one or more of: delay in tumor growth, reduction in tumor cell number, tumor regression, prevention, or delay of tumor recurrence, increase in survival, partial response, and complete response. In some embodiments, the tumor growth in the patient is delayed by at least 10 days as compared to tumor growth in an untreated patient. In some embodiments, the tumor growth is inhibited by at least 20% (e.g., at least 30%, at least 40%, at least 50%, at least 60%, at least 70%, at least 80%, at least 90%, at least 100%, at least 200%, at least 300%) as compared to an untreated patient.

A pharmaceutical composition comprising a LtxA polypeptide can be delivered intratumorally, intravesically, subcutaneously, intraperitoneally, or intravenously, e.g., with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered, and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

In some embodiments, the pharmaceutical composition can be delivered in a controlled release system. In one embodiment, a pump may be used. In another embodiment, polymeric materials can be used; see, e.g., Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous, and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

### Additional Definitions

To aid in understanding the detailed description of the compositions and methods according to the disclosure, a few express definitions are provided to facilitate an unambiguous disclosure of the various aspects of this disclosure. Unless otherwise defined, all technical and scientific terms used herein have the same meaning as commonly understood by one of ordinary skill in the art to which this disclosure belongs.

The term “recombinant,” as used herein, refers to LtxA polypeptides of this disclosure created, expressed, isolated or obtained by technologies or methods known in the art as recombinant DNA technology which include, e.g., DNA splicing and transgenic expression. The term refers to antibodies expressed in a non-human mammal (including transgenic non-human mammals, e.g., transgenic mice), or a cell (e.g., CHO cells) expression system or isolated from a recombinant combinatorial human antibody library.

A “nucleic acid” or “polynucleotide” refers to a DNA molecule (for example, but not limited to, a cDNA or genomic DNA) or an RNA molecule (for example, but not limited to, an mRNA), and includes DNA or RNA analogs. A DNA or RNA analog can be synthesized from nucleotide analogs. The DNA or RNA molecules may include portions that are not naturally occurring, such as modified bases, modified backbone, deoxyribonucleotides in an RNA, etc. The nucleic acid molecule can be single-stranded or double-stranded.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, and more preferably at least about 95%, 96%, 97%, 98% or 99% of the nucleotide bases, as measured by any well-known algorithm of sequence identity, such as FASTA, BLAST or GAP, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As used herein, the term “disease” is intended to be generally synonymous and is used interchangeably with, the terms “disorder” and “condition” (as in medical condition), in that all reflect an abnormal condition (e.g., inflammatory disorder) of the human or animal body or of one of its parts that impairs normal functioning, is typically manifested by distinguishing signs and symptoms, and causes the human or animal to have a reduced duration or quality of life.

The terms “decreased,” “reduced,” “reduction,” “decrease,” or “inhibit” are all used herein generally to mean a decrease by a statistically significant amount. However, for avoidance of doubt, “reduced,” “reduction” or “decrease” or “inhibit” means a decrease by at least 10% as compared to a reference level, for example, a decrease by at least about 20%, or at least about 30%, or at least about 40%, or at least about 50%, or at least about 60%, or at least about 70%, or at least about 80%, or at least about 90% or up to and including a 100% decrease (e.g., absent level as compared to a reference sample), or any decrease between 10-100% as compared to a reference level.

As used herein, the term “agent” denotes a chemical compound, a mixture of chemical compounds, a biological macromolecule (such as a nucleic acid, an antibody, a protein, or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

As used herein, the terms “therapeutic agent,” “therapeutic capable agent,” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder, or condition; and generally counteracting a disease, symptom, disorder, or pathological condition.

The term “therapeutic effect” is art-recognized and refers to a local or systemic effect in animals, particularly mammals, and more particularly humans caused by a pharmacologically active substance.

Doses are often expressed in relation to bodyweight. Thus, a dose which is expressed as [g, mg, or other unit]/kg (or g, mg etc.) usually refers to [g, mg, or other unit]“per kg (or g, mg etc.) bodyweight,” even if the term “bodyweight” is not explicitly mentioned.

As used herein, the term “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained.

As used herein, the term “pharmaceutically acceptable carrier” includes a pharmaceutically acceptable salt, pharmaceutically acceptable material, composition, or carrier, such as a liquid or solid filler, diluent, excipient, solvent, or encapsulating material, involved in carrying or transporting a compound(s) of the present disclosure within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation and not injurious to the subject. Some examples of materials that may serve as pharmaceutically acceptable carriers include: sugars, such as lactose, glucose, and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of one or more components of this disclosure and are physiologically acceptable to the subject. Supplementary active compounds may also be incorporated into the compositions.

“Combination” therapy, as used herein, unless otherwise clear from the context, is meant to encompass administration of two or more therapeutic agents in a coordinated fashion and includes, but is not limited to, concurrent dosing. Specifically, combination therapy encompasses both co-administration (e.g., administration of a co-formulation or simultaneous administration of separate therapeutic compositions) and serial or sequential administration, provided that administration of one therapeutic agent is conditioned in some way on the administration of another therapeutic agent. For example, one therapeutic agent may be administered only after a different therapeutic agent has been administered and allowed to act for a prescribed period of time. See, e.g., Kohrt et al. (2011) Blood 117:2423.

As used herein, “administering” refers to the physical introduction of a composition comprising a therapeutic agent to a subject, using any of the various methods and delivery systems known to those skilled in the art. Example routes of administration for antibodies described herein include intravenous, intraperitoneal, intramuscular, subcutaneous, intratumoral, intravesical, spinal or other parenteral routes of administration, for example, by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, an antibody described herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

As used herein, the term “co-administration” or “co-administered” refers to the administration of at least two agent(s) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

As used herein, the singular forms “a,” “an,” and “the” include plural references unless the context clearly dictates otherwise.

As used herein, the terms “including,” “comprising,” “containing,” or “having” and variations thereof are meant to encompass the items listed thereafter and equivalents thereof as well as additional subject matter unless otherwise noted.

As used herein, the phrases “in one embodiment,” “in various embodiments,” “in some embodiments,” and the like are used repeatedly. Such phrases do not necessarily refer to the same embodiment, but they may unless the context dictates otherwise.

As used herein, the terms “and/or” or “/” means any one of the items, any combination of the items, or all of the items with which this term is associated.

As used herein, the word “substantially” does not exclude “completely,” e.g., a composition which is “substantially free” from Y may be completely free from Y. Where necessary, the word “substantially” may be omitted from the definition of this disclosure.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%1, 1%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percent, proximate to the recited range that are equivalent in terms of the functionality of the individual ingredient, the composition, or the embodiment.

As disclosed herein, a number of ranges of values are provided. It is understood that each intervening value, to the tenth of the unit of the lower limit, unless the context clearly dictates is otherwise, between the upper and lower limits of that range is also specifically disclosed. Each smaller range between any stated value or intervening value in a stated range and any other stated or intervening value in that stated range is encompassed within the disclosure. The upper and lower limits of these smaller ranges may independently be included or excluded in the range, and each range where either, neither, and/or both limits are included in the smaller ranges is also encompassed within the disclosure, subject to any specifically excluded limit in the stated range. Where the stated range includes one or both of these limits, ranges excluding either or both of those included limits are also included in the disclosure.

The use of all examples, or exemplary language (e.g., “such as”) provided herein, is intended merely to better illuminate the invention and does not pose a limitation on the scope of this disclosure unless otherwise claimed. No language in the specification should be construed as indicating any non-claimed element as essential to the practice of this disclosure.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise. In cases in which a method comprises a combination of steps, each and every combination or sub-combination of the steps is encompassed within the scope of this disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present disclosure. Nothing herein is to be construed as an admission that the present disclosure is not entitled to antedate such publication by virtue of prior disclosure. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### I. EXAMPLES

### Example 1

### Composition

The composition of this drug product, i.e., Leukothera, includes: (a) Leukotoxin proteins isolated from a bacterium, as an active pharmaceutical ingredient; (b) Tromethamine (Tris) as buffer; (c) Sodium Chloride (NaCl) Stabilizer; and Calcium Chloride (CaCl₂) Stabilizing agent for activity.

This composition is according to an in-house developed method and calculated weight based on a target to achieve a final concentration of Leukotoxin at 0.30 mg/mL when reconstituted in sterile water at the time of clinical use/infusion. Leukothera for Injection is a white, sterile, lyophilized powder. It is supplied in a clear glass vial and intended to be reconstituted with 2 mL of sterile water at the time of use for intravenous injection. After reconstitution, the vial is gently swirled until all powder has gone into solution. Appropriate volumes for each dose are then added to an IV bag for slow infusion/injection. The concentration of Leukotoxin in Leukothera for Injection is 0.30 mg/mL (300 ug/mL).

Leukothera for Injection is a lyophilized powder of the drug substance, Leukotoxin. Leukotoxin is formulated into a bulk solution in a buffer composed of 20 mM Tromethamine (Tris), 250 mM Sodium Chloride (NaCl), 0.2 mM Calcium Chloride (CaCl₂) at pH 7.5. It should be noted that the formulating occurs as part of drug substance manufacture; no additional formulation step occurs during drug product manufacture. Leukothera for Injection is manufactured to achieve a final concentration of Leukotoxin at 0.30 mg/mL when reconstituted in sterile water at the time of use. The target fill is 0.6 mg of Leukotoxin per vial. The composition of Leukothera for Injection is provided in Table 2.

TABLE 2
Composition of Leukothera for Injection

| | Amount per Vial for | | | |
|---|---|---|---|---|
| | Reconstitutionand | | | Quality |
| Component | Injection | | Function | Standard |
| Leukotoxin (LtxA) | 0.6 | mg | Active | In-house |
| | | | Pharmaceutical | specifi- |
| | | | Ingredient | cation |
| Tromethamine | 4.85 | mg¹ | Buffering agent | USP |
| (Tris) | | | | |
| Sodium Chloride | 29.2 | mg¹ | Stabilizing | USP |
| (NaCl) | | | agent | |
| Calcium Chloride | 0.04 | mg¹ | Stabilizing | USP |
| (CaCl₂) | | | agentfor activity | |

¹Calculated weight based on targeted final concentrations

TABLE 3
Specification of Drug Product, Leukothera for Injection

| Category | Test Items | Methods | Release Criteria | Shelf Life Criteria |
|---|---|---|---|---|
| Quality | Appearance | Visual inspection | White to off-white | White to off-white |
| | (lyophilizate) | | cake, no foreign | cake, no foreign |
| | | | particles visible | particles visible |
| | Appearance | Ph. Eur. 2.2.2 | Clear to slightly | Clear to slightly |
| | (reconstituted | Ph. Eur. 2.2.1 | opalescent and | opalescent and |
| | solution) | Ph. Eur. 2.9.20 | colorless to slightly | colorless to slightly |
| | | | yellow solution, free | yellow solution, free |
| | | | from or practically | from or practically |
| | | | free | free |
| | | | from visible particles | from visible particles |
| | pH | USP <791> | 7.0-8.0 | 7.0-8.0 |
| | Residual | USP <921> 1c | Report results (%) | Report results (%) |
| | moisture | | | |
| Strength | Protein | Total Protein BCA | ≥225 and ≤375 | ≥225 and ≤375 |
| | Concentration | Assay | μg/mL | μg/mL |
| Purity | Protein Purity | CE-SDS | ≥95% purity (based on | ≥95% purity (based |
| | | | the sum of product | onthe sum of product |
| | | | peak areas as a | peak areas as a |
| | | | percentage of all peaks | percentage of all peaks |
| | | | elution from the | elution from the |
| | | | capillary) | capillary) |
| | Protein Purity | RP-HPLC | Main peak ≥90% area | Report results(% main |
| | | | | peak |
| | Charge variants | cIEF | Report results (pI main | Report results (pI main |
| | | | peak) | peak) |
| Potency | Activity | ATP-based Cell | Leukothera mediated | Leukothera mediated |
| | | Viability Assay | cell death ≥50% | cell death ≥50% |
| | | | relative to control, | relative to control, |
| | | | untreated cells | untreated cells |
| Safety | Sterility | USP <71> | Pass per USP <71> | Pass per USP <71>^{a} |
| | | Membrane Filtration | | |
| | Container closure | Dye ingress | N/A | Pass^{b} |
| | Bacterial | USP <85> | Highest measurement | N/A |
| | endotoxin | Recombinant Factor | <0.35 EU/μg protein | |
| | | C Assay | | |

μg = Microgram;
mL = Milliliter
BCA = Bicinchoninic Acid;
CE-SDS = Capillary Electrophoresis-Sodium Dodecyl Sulfate;
RP-HPLC = Reversed Phase High Performance Liquid Chromatography;
cIEF = Capillary Isoelectric Focusing
Ph. Eur. = European Pharmacopoeia;
USP = United States Pharmacopeia;
EU = Endotoxin unit
N/A = Not applicable; distinguishes which test items are evaluated as release only or stability only
^{a}Last stability timepoint only at long term condition only.
^{b}Annual testing at long-term condition only.

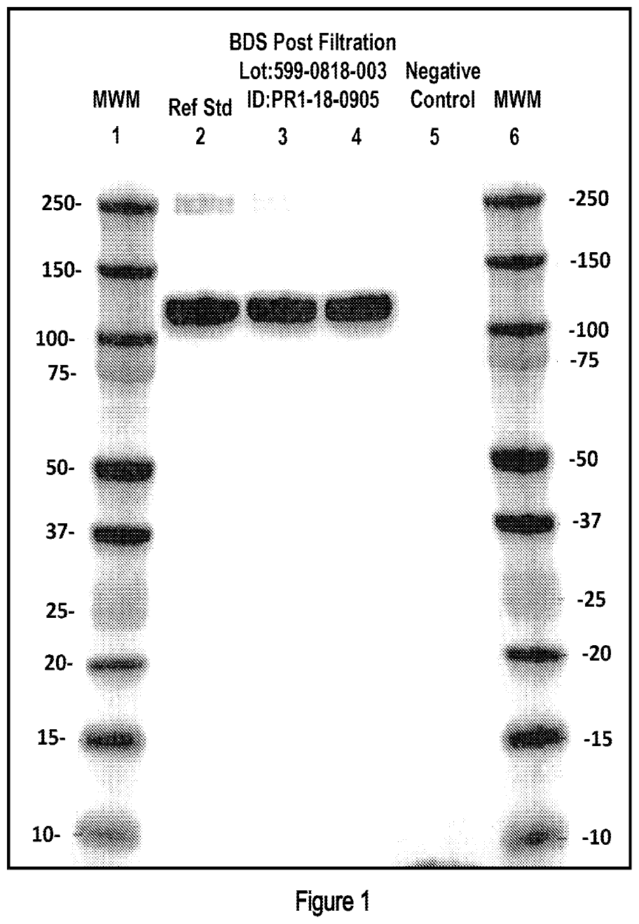

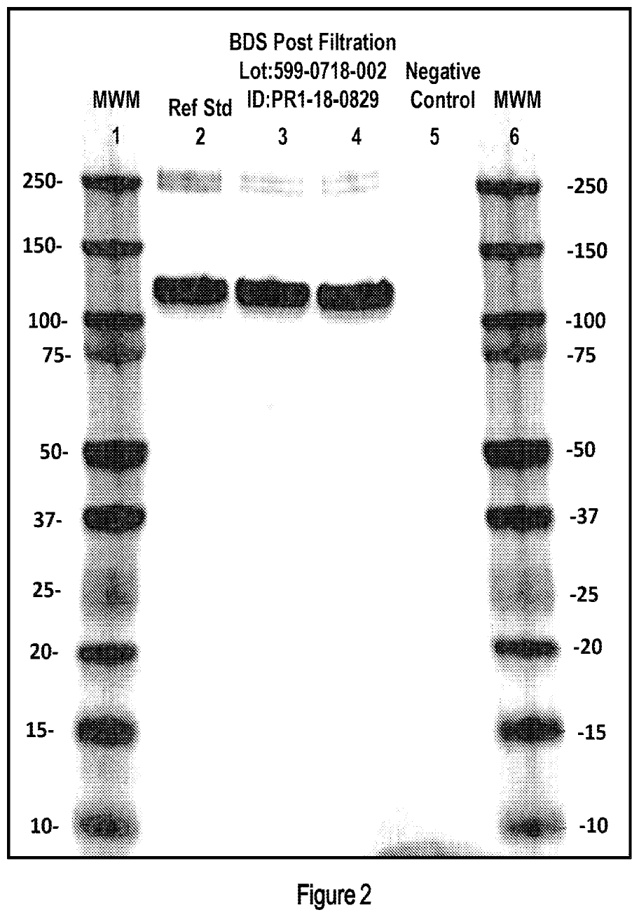

Protein identity can be assessed by qualitative Western Blot. Protein samples are separated on a Sodium Dodecyl Sulfate-Polyacrylamide Gel Electrophoresis (SDS-PAGE) gel through electrophoresis. After SDS-PAGE electrophoresis proteins are transferred to a nitrocellulose membrane. The nitrocellulose membrane is probed with a primary monoclonal antibody raised against the LtxA protein. A conjugated secondary antibody probe binds to the primary monoclonal antibody and is used to detect the proteins and is measured using the ChemDoc MP System with Image Lab Software. The qualitative Western Blot was qualified to demonstrate specificity through the performance of Western Blot on a non-specific protein (bovine serum albumin [BSA]) to determine if any non-specific interaction occurs at the molecular weight of the specific protein. The established acceptance criterion requires observation of major sample band at ˜120 kilodaltons (kDa), conforming to the migration pattern of the reference standard, and ensuring the identity of the protein is confirmed. Data from the Western Blot analysis of the GMP Lot 599-0818-003 and Engineering Lot 599-0718-002 are shown in FIG. 1 and FIG. 2, respectively.

### Formulation

This drug composition disclosed herein is parenteral formulation. The drug product is formulated for injection, for a parenteral route of administration, is a white, sterile, lyophilized powder for solution for infusion, supplied in clear glass vials. Each vial will contain 0.6 mg lyophilized powder. The final dosage form is a solution for intravenous infusion. To obtain the final dosage form, 0.6 mg lyophilized powder will be reconstituted with 2 mL sterile water to a final concentration of 0.3 mg/mL. It is supplied in a clear glass vial and intended to be reconstituted with 2 mL of sterile water at the time of clinical use for intravenous injection. After reconstitution, the vial is gently swirled until all powder has gone into solution. Appropriate volumes for each dose are then added to an IV bag for slow infusion/injection. The infusion concentration of API in this drug product (Leukothera for Injection) is 0.30 mg/mL (300 ug/mL).

### Storage and Stability

The vials containing lyophilized powder must be stored frozen at <−20° C. (standard freezer). After reconstitution with sterile water, the product can be stored at 4° C. (standard refrigeration) for up to 24 hours, after which it must be discarded. All investigational products at the study site must be stored securely locked and with restricted access. Temperature must be controlled during shipment and during storage at study sites.

TABLE 4
Stability Study Summary

| | | | | |
|---|---|---|---|---|
| | | Presentation/ | | Storage |
| Batch ID | Disposition | Strength | Container/Orientation | Condition |

Primary stability

| | | | | |
|---|---|---|---|---|
| 093I0720 | GMP lot | Lyophilizate | 5 mL clear glass vial | −20 ± 5° C. |
| | | 0.7 mg/vial^{a} | 20 mm FluroTec stopper | 5 ± 3° C. |
| | | | Upright | |
| 072I0918 | Engineering | Lyophilizate | 5 mL clear glass vial | −80 ± 10° C. |
| | Lot | 0.6 mg/vial | 20 mm FluroTec stopper | −20 ± 5° C. |
| | | | Upright | |
| 081I1018 | GMP Lot | Lyophilizate | 5 mL clear glass vial | −80 ± 10° C. |
| | | 0.6 mg/vial | 20 mm FluroTec stopper | −20 ± 5° C. |
| | | | Upright | |

Supporting stability

| | | | | |
|---|---|---|---|---|
| 003D0318 | Demo Lot | Lyophilizate | 5 mL clear glass vial | −80 ± 10° C. |
| | | 0.6 mg/vial | 20 mm FluroTec stopper | −20 ± 5° C. |
| | | | Upright | |

^{a}Lot 093I0720 was formulated at 0.7 mg/vial as DS lot A599-LtxA−20-004 had a concentration of353 μg/mL and no further dilution is performed during drug product manufacture.

### Stability at Long Term Storage Condition

At the long-term storage condition of −20±5° C., data are available for primary lots up to 12 months (07210918 and 08111018). Stability data for the supporting stability lot are available for up to 19 months. All lots met all specifications when stored at ≤−20±5° C. through 12 months, and demonstrate that the drug product is stable at this storage condition.

When stored at −20±5° C., data from lot 09310720, employing the Phase 1 specification, do not reveal any trends in quality attributes related to formulation (pH and protein content), biological activity (potency) and purity (CE-SDS, RP-HPLC, and cIEF).

### Clinical In-Use Stability

To mimic the extreme of potential conditions of clinical use, stability (as assessed by activity according to the ATP-based Cell Viability Assay) of Leukothera for Injection was studied following reconstitution under the following clinically relevant conditions:

Refrigeration: Remove lyophilized samples from ≤−20° C., reconstitute, and then store for up to 24 hours at 4° C.

Conclusion: Samples frozen ≤12 months can be stored for up to 24 hours at 4° C.

Refreezing: Remove lyophilized samples from ≤−20° C., reconstitute, and then store for up to 7 days at ≤−20° C.

Conclusion: Lyophilized samples frozen ≤12 months at ≤−20° C. can be reconstituted and frozen again (at ≤−20° C.) for up to 7 days

The results support that there is no significant impact on product quality for Leukothera for Injection following refrigeration or refreezing in the clinical use setting.

### Shelf Life

The primary stability batches support for a shelf-life determination of 18 months for clinical material is based on an appropriate extension of available stability data from the development study of Leukothera for Injection Lot 07210918 (Engineering Lot) and Lot 08111018 (GMP Lot), which met all specifications at −20±5° C. through 12 months. The data from Leukothera for Injection Lot 003D0318, which has been analyzed following 19 months of storage, further supports the 18-month shelf life.

The recommended storage condition for Leukothera for Injection clinical material is ≤−20° C. for a shelf life of 18 months.

### Container Closure System

The primary container closure for Leukothera for Injection is a 5 mL clear glass vial and a 20 mm FluroTec stopper. The vial is sealed with a flip-off TrueEdge seal.

TABLE 5
Primary Container Closure for Leukothera for Injection

| Component | Description | Supplier |
|---|---|---|
| Vial | 5 mL clear glass, 20 mm opening, | West Pharmaceutical |
| | USP Type1 tubing glass | Services |
| Stopper | 20 mm FluroTec chlorobutyl | West Pharmaceutical |
| | stopper | Services |
| Seal | 20 mm flip-off TrueEdge, 8-bridge | West Pharmaceutical |
| | seals withtop button | Services |

### Example 2

### Manufacturing and Process Development

This section describes the manufacturing process for Leukothera for Injection, as conductedat The University of Iowa Pharmaceuticals, for the Good Manufacturing Practice (GMP) Lot 08111018. A manufacturing flow diagram indicating the applicable process controls is provided below.

Leukothera for Injection Manufacturing Process Flow Diagram

### Thawing

Container(s) of Leukothera Bulk Drug Solution (BDS) are removed from frozen storage (−60° C. to −90° C.) and placed upright at refrigerated storage to thaw (2-8° C.). Approximately 3 to 4 days are allowed for thawing time. The thawed solution is removed from the refrigerator and transferred to the compounding area.

### Pooling

Each container of Leukothera Bulk Solution is slowly rotated/swirled and then carefully poured into a clean 9 L glass carboy with a stir bar. The solution is gently mixed with a Magnetic Stirrer until homogeneous (for a minimum of 5 minutes).

### Sterile Filtration

Once the Clean Room is deemed suitable for use by Standard Operating Procedure, the containers, closures, and equipment (standard Hull HY-PRO lyophilizer) are transported into the Clean Room. Non-viable particulates in the room are monitored via a particle sensor.

The Clean Room pressure differential is also monitored and verified to be at least 0.05 inches of water higher than the Entry Room. The 9L glass carboy containing the pooled Leukothera Bulk Solution is then transported to the Clean Room for filtration. The viable flora in the Clean Room are monitored. The pooled Leukothera Bulk Solution is then sterile filtered into a sterile 9 L glass carboy using a sterile 0.22 micron, pore-size Millipak 200 filter. The carboy remains covered with a sterile stopper until the start of the filling process. At the end of filtration, the integrity of the filter is tested according to Standard Operating Procedure.

The water bubble point of 50 PSI should be reached. An initial rinse of the filter is completedPRe using 70/30 IPA/WFI followed by water, and the flush times and bubble point is results arerecorded. A satisfactory bubble point must be obtained prior to beginning the filling operation. If the filter bubble point fails, then filtration must be repeated using a new filter.

A satisfactory bubble point must be obtained prior to beginning the filling operation.

### Filling Vials

Using a sterile Flexicon FP50 Filler, each sterile 5 mL vial is filled with approximately 2.04 grams (g) of filtered Leukothera Bulk Solution. Each vial is stoppered to lyophilization depth in accordance with Standard Operating Procedure. All vials undergo an in-process fill weight check. Vials that are not filled within the specified weight range of 2.04g+/−0.20 g are rejected.

### Lyophilization

Each tray of vials is placed in the lyophilizer as the vials are filled. The lyophilizer is loaded at Room Temperature. The Hull HY-PRO Lyophilizer is used to lyophilize the filled vials of Leukothera Bulk Solution according to the specified cycle:

TABLE 6
Lyophilization Cycle for Leukothera for Injection

| | | Time | Vacuum | |
|---|---|---|---|---|
| Step# | TemDIC\ | (Minutesl | (mTorr) | Ramo/Hold |
| Thermal | | | | |
| Treatment | | | | |
| 1 | −35 | 10 | NA | R |
| 2 | −35 | 20 | NA | H |
| Freeze | −35 | 10 | 100-300 | H |
| Condenser and | | | | |
| Evacuate | | | | |
| Primary Drying | | | | |
| 1 | −30 | 10 | 100 | R |
| 2 | −30 | 1080 | 100 | H |
| 3 | −25 | 10 | 100 | R |
| Secondary | | | | |
| 1 | 0 | 240 | 200 | R |
| 2 | +25 | 300 | 200 | R |
| 3 | +25 | 60 | 200 | H |
| 4 | +25 | Until | NA | H |
| | | stopperina | | |

The vials containing the lyophilized product are held and stored at 25° C.

### Stoppering and Sealing

After the lyophilization cycle is complete, the chamber and vials are purged to atmosphere with Nitrogen, NF. The vials are then stoppered. The vial height of the cap closing station (crimper) is verified. Vials are removed from the chamber and a sterile seal is placed on each vial. The seals are crimped in accordance with Standard Operating Procedure (SOP). Sealed vials of Leukothera for Injection are stored at 2-8° C.

Leukothera for Injection vials undergo a 100% manual visual inspection followed by Acceptable Quality Limit (AQL) sampling inspection, as per The University of Iowa Pharmaceuticals SOP, for particulate matter and flaws in the container-closure system.

### Labeling, Packaning, and Storage

Vials of Leukothera for Injection are labeled with an approved vial label. Labeled vials is are bulk packaged and stored at −70° C. until shipment.

TABLE 7
Description of Investigational Product

| | |
|---|---|
| Product name | Leukothera for Injection |
| Chemical name | Leukotoxin; protein derived from |
| | A. actinomycetemcomitans |
| Dosage form | Injection, Powder, Lyophilized, For Solution |
| Formulation | The investigational product will be supplied |
| | in glassvials containing 0.6 mg lyophilized |
| | powder. The investigational |
| | product will be reconstituted with |
| | 2 mL sterile water to a final concentration of |
| | 0.3 mg/mL at the study sites and administered |
| | as asolution via slow intravenous infusion. |

### Biological Characteristics

The Active Pharmaceutical Ingredient (API) is the Drug Substance, Leukotoxin. Leukotoxin is post-translationally modified at lysine residues K561 and K686 with fatty acyl groups. These acylations are required for Leukotoxin activity against LFA-1 expressing leukocytes. Leukotoxin contains no cysteine residues or glycosylated motifs.

### Example 3

### Posology and Method of Administration

### Target Clinical Dose

Drug product lyophilizate is reconstituted in the vial with 2 mL sterile water to a concentration of 0.3 mg/mL. Reconstituted drug product solution is diluted in 0.9% saline in an IV bag and infused over a period of up to four hours: Low dose: 1.4 μg/kg in a 70 kg subject, or 98 μg 0.25 μg/mL concentration and volume 400 mL. High dose: 1020 μg/kg in a 70 kg subject, or 71.4 mg 0.18 mg/mL concentration and volume 400 mL.

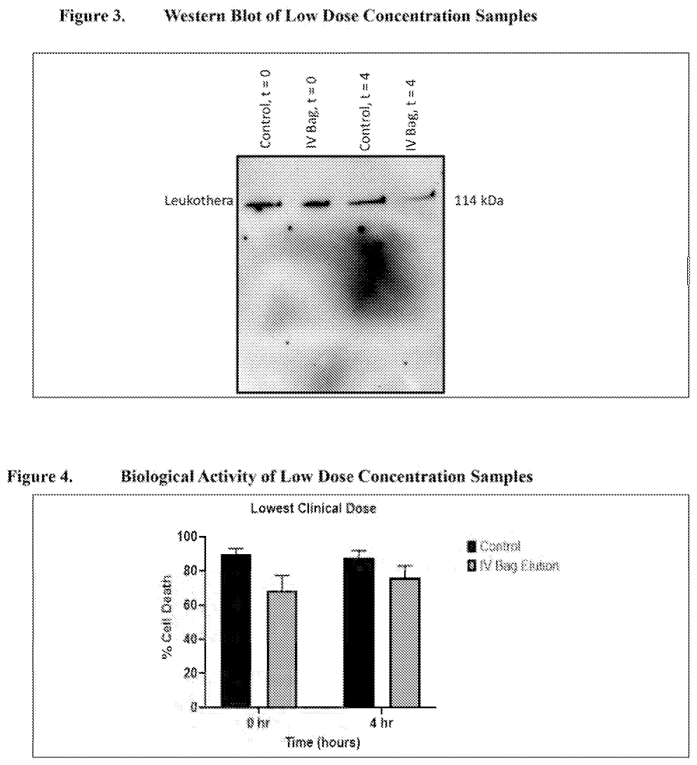

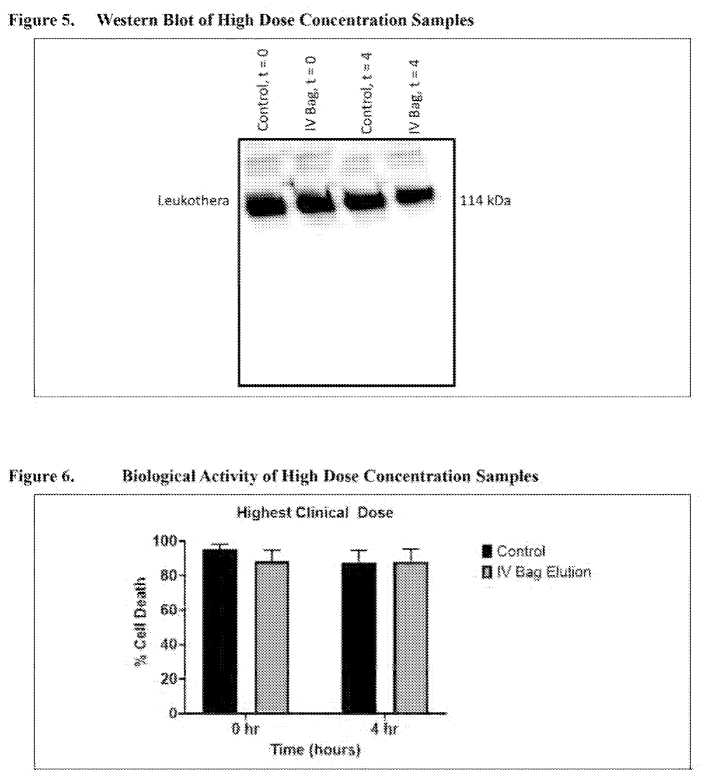

Results for protein concentration in all samples at the low dose concentration were below the limit of quantification for the BCA assay (0.20 g/mL) for total protein content, which reflects that the low dose concentration sample is around this level at 0.25 g/mL. The western blot was used to show that protein was recovered as shown in FIG. 3. Although no purity determination was undertaken by scanning the western blot, there is visually no change in number of bands over time held in the IV bag compared to both T=0 and the control samples. Results of bioactivity at the low dose concentration demonstrate that active protein content was recovered that effects cell death at levels within the expected drug product release range (50-150%), as shown in FIG. 4. Similar results were seen for the highest dose as shown in FIG. 5 and FIG. 6. Again, there was no change in types of bands seen in the western blot between the test sample and the T=0 and control samples indicating that there was no loss in purity over the 4 hour hold time.

TABLE 8
Administration Parameters for Infusion Bag Compatibility Study

| | Clinical | | |
|---|---|---|---|
| Parameter | Administration | Study | Comment |
| Solution for | Sterile WFI | Sterile WFI | Same |
| reconstitution | | | |
| Infusion bagmodel/type: | Commercially | Baxter | |
| | available | | |
| Plastic | Commercially | Polyvinylchloride(PL | PVC representativeof |
| | available | 146) | commercial bag |
| Size | 500 mL bag | 50 mL bag | 1:10 scale |
| | 500 mL nominal | 50 mL nominal | |
| | volume | volume | |
| Solution | Clinical saline | 0.9% Sodium | Same |
| | | Chloride InjectionUSP | |
| In-line filter | 0.2 μm | 0.2 μm | Same |
| Low dose concentration 0.25 μg/mL | | | Concentration: same |
| Saline added | 450 mL | 45 mL | Volume to surface |
| Reconstituted drug | 420 μL diluted | 42 μL DP dilutedup to | area: Same as 1:10 |
| product added | up to 50 mL saline | 5 mL saline | scale for both bag size |
| Volume in bag | 500 mL | 50 mL | and volume |
| High dose concentration 0.18 mg/mL | | | Concentration: same |
| Saline added | 200 mL | 4 mL | Volume to surface |
| Reconstituted drug | 300 mL DP | 6 mL DP | area: 1:50 scale |
| product added | | | volume in 1:10 scale |
| Volume in bag | 500 mL | 10 mL | bag, worstcase is low |
| | | | doseconcentration |

Infusion

| | | | |
|---|---|---|---|
| Duration/exposure tobag | Up to 4 hours | 4 hours | Worst case |

TABLE 9
Results of Analytical Testing of Low Dose Concentration
Leukothera (0.25 μg/mL) In IV Bag Compatibility Study

| | | | | |
|---|---|---|---|---|
| | Protein | Protein | | |
| | Concen- | Concen- | Bioactivity | |
| | tration | tration | (% cell | |
| | by BCA | byWestern | death, mean | |
| Sample | (μg/mL) | blot (μg/mL) | of n = 6) | Appearance |

t = 0 hours

| | | | | |
|---|---|---|---|---|
| Control | <LOQ | 0.25 | Mean 90; SD: | Clear/colorless to |
| | | | 3 (90, 90, 89 | slightlyopalescent; |
| | | | 89, 94, 83) | free of particulates |
| IV Bag | <LOQ | 0.214 | Mean 70; SD: | Clear/colorless to |
| | | | 9 (69, 61,70, | slightlyopalescent; |
| | | | 71, 57, 82) | free of particulates |

t = 4 hours

| | | | | |
|---|---|---|---|---|
| Control | <LOQ | 0.215 | Mean 85; SD | Clear/colorless to |
| | | | 8 (89, 89, 89, | slightlyopalescent; |
| | | | 81, 92, 82) | free of particulates |
| IV Bag | <LOQ | 0.093 | Mean 71; SD | Clear/colorless to |
| | | | 14 (78, 84, 79, | slightlyopalescent; |
| | | | 64, 71, 77) | free of particulates |

LOQ—limit of quantification (20 μg/mL)

TABLE 10
Results of Analytical Testing of High Dose Concentration
Leukothera In IV Bag (0.18 mg/mL) Compatibility Study

| | | | | |
|---|---|---|---|---|
| | Protein | Protein | | |
| | Concen- | Concen- | Bioactivity | |
| | tration | tration | (% cell | |
| | by BCA | byWestern | death, mean | |
| Sample | (μg/mL) | blot (μg/mL) | of n = 6) | Appearance |

t = 0 hours

| | | | | |
|---|---|---|---|---|
| Control | 160 | 210 | Mean 95; SD | Clear/colorless to |
| | | | 1 (93, 92, 92, | slightlyopalescent; |
| | | | 97, 98, 98) | free of particulates |
| IV Bag | 148 | 212 | Mean 89; SD | Clear/colorless to |
| | | | 1 (82, 84, 83, | slightlyopalescent; |
| | | | 95, 95, 95) | free of particulates |

t = 4 hours

| | | | | |
|---|---|---|---|---|
| Control | 152 | 172 | Mean 88; SD | Clear/colorless to |
| | | | 1 (81, 80, 82, | slightlyopalescent; |
| | | | 92, 93, 95) | free of particulates |
| IV Bag | 115 | 110 | Mean 88; SD | Clear/colorless to |
| | | | 4 (80, 88, 78, | slightlyopalescent; |
| | | | 90, 92, 97) | free of particulates |

The low dose for the human Phase 1 study was selected based on the 10%-maximal effective concentration (EC10), calculated from an in vitro model using THP-1 cells, a human monocytic cell line derived from an acute monocytic leukemia patient. Diseased WBCs have a higher expression of LFA-1, the target of Leukothera for injection, when compared to healthy WBCs. Therefore, the EC10 estimate from the in vitro model using THP-1 cells derived from a patient with acute monocytic leukemia will be most representative of an EC10 in patients being treated with Leukothera for Injection. The calculated EC10 from the in vitro model was 20 ng/mL. In a 70-kg human with blood volume of approximately 5 L, a dose of 1.4 μg/kg is predicted to result in Leukothera for Injection concentrations approximating the EC10. Thus, 1.4 μg/kg/week is the proposed starting dose in humans and will be administered as a single IV dose initially over the course of 3-4 hours with the intent of dosing weekly and the flexibility of modifying the duration of the IV infusion, if needed. Additional support for the proposed starting dose of 1.4 μg/kg comes from ICH S9 and the standard approach for estimating the starting dose in initial clinical trials for anticancer pharmaceuticals, 1/10 the STD10 in rodents or ⅙ the HNSTD in non-rodents on a body surface area (BSA) normalized basis.

Although Leukothera for Injection is a biologic, the calculations were conducted to aid in the selection of an appropriate starting dose in humans. The STD10 in male rats in the 4-week study was between 500 and 750 μg/kg while the STD10 in female rats was 1000 μg/kg. Using the most conservative dose level of 500 μg/kg, which was the NOAEL, the human starting dose is estimated as follows: ⋅NOAEL in rat=500 μg/kg÷1000 μg=0.5 mg/kgψBSA-normalized Human Equivalent Dose=0.5 mg/kg÷6.2=0.08 mg/kg×1000=81 μg/kg⋅Human starting dose (BSA-normalized): 81 μg/kg÷10 (safety factor)=8.1 μg/kg Based on the HNSTD of 300 μg/kg in the 4-week dog study, which was also a NOAEL, the human starting dose is estimated as follows: ⋅NOAEL in dog=300 μg/kg÷1000 μg=0.3 mg/kg⋅BSA-normalized Human Equivalent Dose=0.3 mg/kg÷1.8=0.167 mg/kg×1000=167 μg/kg⋅Human starting dose (BSA-normalized): 167 μg/kg÷6 (safety factor)=27.8 μg/kg Thus, the most conservative starting dose on a BSA normalized basis would be 8.1 μg/kg. With the proposed starting dose of 1.4 μg/kg, this is >5-fold lower than the recommended starting dose on a BSA-normalized basis.

The rat was the most sensitive nonclinical species, and the proposed human starting dose is 57-fold lower than the body surface area (BSA)-normalized human dose at the lower end of the STD10 range (500 μg/kg), and 86-fold lower than the human equivalent dose (HED) associated with mortality in rats (750 mg/kg). The HNSTD in dogs was the highest dose level tested (300 μg/kg), or 119 times higher than the proposed starting dose on a BSA-normalized basis. Transient elevations in various cytokines indicate that there is a potential for infusion-related cytokine release in patients, such that patients should be monitored appropriately with treatment implemented if necessary.

Additionally, there is an ongoing veterinary clinical study in canines with lymphoma in which 8 dogs have been administered once weekly escalating doses of Leukothera for Injection intravenously over 30 minutes at dose levels ranging from 5 to 200 μg/kg. None of the dogs has experienced any adverse reactions during or after infusion with Leukothera for Injection. On a BSA-normalized basis, this dose range is equivalent to 2.8-111 μg/kg in humans and is 2- to 79-fold higher than the proposed human starting dose of 1.4 μg/kg.

### Preparation and Administration Procedures

Each site will be provided with a Pharmacy Manual, which will include detailed reconstitution and preparation instructions of the investigational product. The product will be reconstituted in 2 mL sterile water to achieve a dosage strength of 0.3 mg/mL. The solution will be transferred to an infusion bag and pump and adjusted with saline to obtain the selected dose. The maximum hold time for the reconstituted product is 24 hours at 4° C. (standard refrigeration), after which it must be discarded. The investigational product will be administered by slow intravenous infusion. The first infusion of investigational product at any dose for a patient will be given over 3-4 hours (+/−15 minutes). If the patient has not experienced an adverse reaction, subsequent infusions at a previously tolerated dose level may be given over 1-2 hours (+/−15 minutes). The infusion time must not be less than 1 hour for any infusion.

The present disclosure is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

What is claimed is:

1.-32. (canceled)

33. A liquid composition for treatment of cancer, comprising:
about 0.1 mg/ml to about 0.5 mg/ml of a leukotoxin (LtxA) polypeptide isolated from Aggregatibacter actinomycetemcomitans,
about 5 mM to about 50 mM Tris,
about 100 mM to about 300 mM NaCl, and
about 0.05 mM to about 0.5 mM CaCl₂,
wherein the liquid composition is formulated to a pH of about 7.0 to about 8.0.

34. The liquid composition of claim 33, comprising about 0.3 mg/ml of the LtxA polypeptide.

35. The liquid composition of claim 33, comprising about 20 mM Tris, about 250 mM NaCl, and about 0.2 mM CaCl₂.

36. The liquid composition of claim 33, wherein the liquid composition is formulated to a pH of about 7.5.

37. The liquid composition of claim 33, wherein the liquid composition is formulated to remain stable for at least 24 hours at 4° C.

38. The liquid composition of claim 33, wherein the LtxA polypeptide is isolated from the NJ4500 strain of Aggregatibacter actinomycetemcomitans.

39. The liquid composition of claim 33, wherein the LtxA polypeptide comprises an amino acid sequence having at least 90% identity to SEQ ID NO: 1 or comprises the amino acid sequence of SEQ ID NO: 1.

40. A lyophilized composition prepared from the liquid composition of claim 33, wherein the lyophilized composition comprises:
about 0.2 mg to about 2 mg of the LtxA polypeptide,
about 2 mg to about 8 mg of Tris,
about 10 mg to about 50 mg of NaCl, and
about 0.01 mg to about 0.5 mg CaCl₂,
wherein the lyophilized composition is formulated to have when reconstituted a pH of about 7.0 to 8.0.

41. The lyophilized composition of claim 40, wherein the lyophilized composition comprises:
about 0.6 mg of the LtxA polypeptide,
about 4.85 mg of Tris,
about 29.2 mg of NaCl, and
about 0.04 mg of CaCl₂.

42. The lyophilized composition of claim 40, wherein the lyophilized composition is formulated to have a pH of about 7.5 after reconstitution.

43. The lyophilized composition of claim 40, wherein the lyophilized composition is reconstituted in sterile water or a saline buffer.

44. The lyophilized composition of claim 40, wherein the lyophilized composition is reconstituted as a liquid composition comprising about 0.3 mg/ml of the LtxA polypeptide.

45. The lyophilized composition of claim 40, wherein the lyophilized composition is formulated to remain stable after storage at −20±5° C. up to 24 months.

46. The lyophilized composition of claim 40, wherein the lyophilized composition is formulated to remain stable after storage at a temperature lower than −20° C., reconstitution, and then storage for up to 7 days at the temperature lower than −20° C.

47. The lyophilized composition of claim 40, wherein the lyophilized composition is formulated to remain stable after storage at a temperature lower than −20° C., reconstitution, and then storage for up to 24 hours at about 4° C.

48. A kit comprising the liquid composition of claim 33.

49. A method for cancer in a subject, comprising administering to the subject a therapeutically effective amount of the liquid composition of claim 33, wherein the therapeutically effective amount of the liquid composition is about 1 μg/kg to about 1200 μg/kg based on weight of the subject.

50. The method of claim 49, wherein the therapeutically effective amount of the liquid composition is about 1.4 μg/kg based on weight of the subject.

51. The method of claim 49, wherein the liquid composition is administered parenterally to the subject intravenously, subcutaneously, or intraperitoneally.

52. The method of claim 49, wherein the liquid composition is administered to the subject by intravenous infusion over a period of 1 to 10 hours.
